(19) United States
(12) Patent Application Publication
(10) Pub. No.: US 2021/0338740 A1
(43) Pub. Date: Nov. 4, 2021

(54) THERAPEUTIC METHODS AND COMPOSITIONS

(71) Applicant: PLURISTEM LTD., Haifa (IL)

(72) Inventors: Zami ABERMAN, Tel-Mond (IL); Yaacob YANAY, Shimshit (IL); Rachel OFIR, Adi (IL); Marc TRITEL, Bet Shemesh (IL); Hoshea Yissachar ALLEN, Bet Shemesh (IL); Niva SHRAGA HELED, Nahariya (IL)

(73) Assignee: PLURISTEM LTD., Haifa (IL)

(21) Appl. No.: 17/283,026

(22) PCT Filed: Nov. 6, 2019
(86) PCT No.: PCT/IB2019/059544

§ 371 Date: Apr. 6, 2021

Related U.S. Application Data
(60) Provisional application No. 62/757,222, filed on Nov. 8, 2018.

(51) Int. Cl. A61K 35/50

(52) U.S. Cl. CPC A61K 35/50

(57) ABSTRACT

Disclosed herein are pharmaceutical compositions and methods comprising or utilizing placental adherent stromal cells. The pharmaceutical compositions may be indicated for ameliorating or treating various disorders, e.g., impaired glucose tolerance. Alternatively, the pharmaceutical composition may be indicated for ameliorating or treating systemic inflammation, e.g., in a subject with impaired glucose tolerance. The pharmaceutical compositions may further include pharmacologically acceptable excipients.

### FIELD

Disclosed herein are therapeutic methods and compositions utilizing placental stromal cells.

### BACKGROUND

Metabolic syndrome, also known as syndrome X or dysmetabolic syndrome, refers to a cluster of metabolic conditions that can lead to heart disease, retinopathy, nephropathy, neuropathy, and a host of other sequelae. Features of metabolic syndrome include insulin resistance, hypertension, an abnormal cholesterol profile, and an increased risk for clotting. People diagnosed with this syndrome are usually overweight or obese. Insulin resistance is a condition in which the body produces insulin but does not respond to it properly. Improved therapies for these conditions are urgently needed in the art.

### SUMMARY

Provided herein are methods of ameliorating and treating elevated blood glucose levels, Impaired Glucose tolerance (IGt), systemic inflammation, and sequelae thereof, in subjects with obesity and metabolic disorders, such as T2DM and metabolic syndrome, comprising administration of adherent stromal cells (ASC). In certain embodiments, the ASC are derived from a placenta. In other embodiments, the ASC are derived from adipose tissue, or BM. In other embodiments, the ASC are derived from a different source tissue.

In certain embodiments, the ASC described herein have been cultured on a 2-dimensional (2D) substrate, a 3-dimensional (3D) substrate, or a combination thereof. Non-limiting examples of 2D and 3D culture conditions are provided in the Detailed Description and in the Examples.

Reference herein to “growth” of a population of cells is intended to be synonymous with expansion of a cell population.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 depicts the study visit flow chart for the study described in Example 5.

FIG. 3 is a chart showing characteristics of placental ASC expanded on 2D substrates, followed by 3D carrier expansion and removal from the carriers. CV % indicates the coefficient of variance, obtained by dividing the standard deviation by the average, and multiplying×100.

FIGS. 4A-C are charts showing stimulation of endothelial cell proliferation and VEGF secretion by ASC (A-B), and IL-10 secretion by monocytes coincubated with ASC (C) for 3 representative batches of placental ASC expanded as described for FIG. 3. For A and C, the vertical axis is percentage activity of the reference batch; while for B, the vertical axis shows picograms per milliliter (pg/ml) of VEGF.

FIGS. 5A-C are charts showing percent viability (A), percent recovery (B) and percent of cell adhesion (C) of the 3 representative batches examined in the previous figure.

FIG. 6 contains charts depicting the mean (A) and adjusted mean (B) log MWD change of subjects in the FAS2Rx receiving placebo (dashed line) or 2 injections of 300 million ASC from 2 different placentas (dotted line) or the same placenta (solid line). Bars depict the standard error.

FIG. 7 is a plot showing change from baseline in blood CRP (nmol/L) in subjects who received 2 doses of 300M ASC each from a different placenta (dotted line) or from the same placenta (dashed line), and the PBO-PBO group (solid line). All subjects were from the mFAS population. Vertical axis: adjusted means+/−SE of change in blood CRP (nmol/L). Horizontal axis: study week. One asterisk (*) indicates p=0.040 (ASC from 1 donor vs ASC from 2 donors), whereas two asterisks (**) indicate p=0.038 (ASC from 1 donor vs ASC from 2 donors) and p=0.0012 (PBO-PBO vs ASC from 2 donors).

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise placental adherent stromal cells (ASC). In certain embodiments, the cells are allogeneic. In other embodiments, the cells may be autologous. Alternatively or in addition, the cells may be fresh or, in other embodiments, frozen (for example, cryo-preserved). Allogeneic, as used herein (except where indicated otherwise), refers to a biological material (e.g. ASC) not derived from, and not syngeneic with, the subject being treated. Typically, allogeneic ASC are neither syngeneic nor haploidentical with the subject. In some embodiments, the described ASC are allogeneic human ASC.

In some embodiments, there is provided a method of ameliorating Impaired Glucose tolerance (IGt) in a subject in need thereof, comprising: administering to the subject a pharmaceutical composition, comprising placental ASC, thereby ameliorating IGt. In other embodiments, there is a provided a pharmaceutical composition comprising placental ASC, for ameliorating IGt. In other embodiments, there is provided use of placental ASC in the preparation of a medicament for ameliorating IGt. In other embodiments, there is provided an article of manufacture, comprising a packaging material and a pharmaceutical composition comprising placental ASC and identified for ameliorating IGt, the pharmaceutical compositions being contained within the packaging material. In some embodiments, the indication is specified in a leaflet that is included within the article of manufacture. In other embodiments, there is provided ASC for use in a method of ameliorating IGt.

In other embodiments, there is provided a method of reducing blood sugar levels of a subject with IGt, comprising: administering to the subject a pharmaceutical composition, comprising placental ASC, thereby reducing blood sugar levels of a subject with IGt. In other embodiments, there is a provided a pharmaceutical composition comprising placental ASC, for reducing blood sugar levels of a subject with IGt. In other embodiments, there is provided use of placental ASC in the preparation of a medicament for reducing blood sugar levels of a subject with IGt. In other embodiments, there is provided an article of manufacture, comprising a packaging material and a pharmaceutical composition comprising placental ASC and identified for ameliorating IGt, the pharmaceutical compositions being contained within the packaging material. In some embodiments, the indication is specified in a leaflet that is included within the article of manufacture.

In other embodiments, there is provided a method of reducing blood sugar levels of a subject with Type 2 Diabetes Mellitus (T2DM), comprising: administering to the subject a pharmaceutical composition, comprising placental ASC, thereby reducing blood sugar levels of a subject with T2DM. In other embodiments, there is a provided a pharmaceutical composition comprising placental ASC, for reducing blood sugar levels of a subject with T2DM. In other embodiments, there is provided use of placental ASC in the preparation of a medicament for reducing blood sugar levels of a subject with T2DM. In other embodiments, there is provided an article of manufacture, comprising a packaging material and a pharmaceutical composition comprising placental ASC and identified for reducing blood sugar levels of a subject with T2DM, the pharmaceutical compositions being contained within the packaging material. In some embodiments, the indication is specified in a leaflet that is included within the article of manufacture.

In still other embodiments, there is provided a method of reducing an incidence of retinopathy in a subject with elevated glucose levels, comprising: administering to the subject a pharmaceutical composition comprising placental ASC, thereby reducing an incidence of retinopathy in a subject with elevated glucose levels. In other embodiments, there is a provided a pharmaceutical composition comprising placental ASC, for reducing an incidence of retinopathy in the aforementioned subject. In other embodiments, there is provided use of placental ASC in the preparation of a medicament for reducing an incidence of retinopathy in the aforementioned subject. In other embodiments, there is provided an article of manufacture, comprising a packaging material and a pharmaceutical composition comprising placental ASC and identified for reducing an incidence of retinopathy in the aforementioned subject, the pharmaceutical compositions being contained within the packaging material. In some embodiments, the indication is specified in a leaflet that is included within the article of manufacture. In certain embodiments, the subject exhibits IGt. In other embodiments, the subject exhibits T2DM. In certain embodiments, an incidence of proliferative retinopathy is reduced. In other embodiments, an incidence of severe nonproliferative retinopathy is reduced. Nathan D M et al. shows that reducing glucose levels prevents retinopathy. Methods of diagnosing retinopathy are known in the art, and are described, for example, in Biyani R S et al and the references cited therein.

In still other embodiments, there is provided a method of reducing an incidence of nephropathy in a subject with elevated glucose levels, comprising: administering to the subject a pharmaceutical composition, comprising placental ASC, thereby reducing an incidence of nephropathy in the aforementioned subject. In other embodiments, there is a provided a pharmaceutical composition comprising placental ASC, for reducing an incidence of nephropathy in the aforementioned subject. In other embodiments, there is provided use of placental ASC in the preparation of a medicament for reducing an incidence of nephropathy in the aforementioned subject. In other embodiments, there is provided an article of manufacture, comprising a packaging material and a pharmaceutical composition comprising placental ASC and identified for reducing an incidence of nephropathy in the aforementioned subject, the pharmaceutical compositions being contained within the packaging material. In some embodiments, the indication is specified in a leaflet that is included within the article of manufacture. In various embodiments, the nephropathy is characterized by microalbuminuria (urinary albumin excretion of at least 40 mg per 24 hours), or albuminuria (urinary albumin excretion of at least 300 mg per 24 hours) In certain embodiments, the subject exhibits IGt. In other embodiments, the subject exhibits T2DM. Nathan D M et al. shows that reducing glucose levels prevents nephropathy. Methods of diagnosing nephropathy are known in the art, and are described, for example, in Umanath K et al. and the references cited therein.

In still other embodiments, there is provided a method of reducing an incidence of neuropathy in a subject with elevated glucose levels, comprising: administering to the subject a pharmaceutical composition, comprising placental ASC, thereby reducing an incidence of neuropathy in the aforementioned subject. In other embodiments, there is a provided a pharmaceutical composition comprising placental ASC, for reducing an incidence of neuropathy in the aforementioned subject. In other embodiments, there is provided use of placental ASC in the preparation of a medicament for reducing an incidence of neuropathy in the aforementioned subject. In other embodiments, there is provided an article of manufacture, comprising a packaging material and a pharmaceutical composition comprising placental ASC and identified for reducing an incidence of neuropathy in the aforementioned subject, the pharmaceutical compositions being contained within the packaging material. In some embodiments, the indication is specified in a leaflet that is included within the article of manufacture. In various embodiments, the neuropathy is peripheral neuropathy, or in other embodiments autonomic neuropathy. In certain embodiments, the subject exhibits IGt. In other embodiments, the subject exhibits T2DM. Nathan D M et al. shows that reducing glucose levels prevents neuropathy. Methods of diagnosing peripheral and autonomic neuropathy are known in the art, and are described, for example, in the article titled “Factors in development of diabetic neuropathy. Baseline analysis of neuropathy in feasibility phase of Diabetes Control and Complications Trial (DCCT). The DCCT Research Group” (Diabetes. 1988 April; 37(4):476-81. PMID: 2897940) and the references cited therein.

In still other embodiments, there is provided a method of reducing an incidence of respiratory disorders in a subject with elevated glucose levels (or in other embodiments, with systemic inflammation; or in other embodiments, with systemic inflammation that accompanies metabolic syndrome), comprising: administering to the subject a pharmaceutical composition, comprising placental ASC, thereby reducing an incidence of respiratory disorders in the aforementioned subject. In other embodiments, there is a provided a pharmaceutical composition comprising placental ASC, for reducing an incidence of respiratory disorders in the aforementioned subject. In other embodiments, there is provided use of placental ASC in the preparation of a medicament for reducing an incidence of respiratory disorders in the aforementioned subject. In other embodiments, there is provided an article of manufacture, comprising a packaging material and a pharmaceutical composition comprising placental ASC and identified for reducing an incidence of respiratory disorders in the aforementioned subject, the pharmaceutical compositions being contained within the packaging material. In some embodiments, the indication is specified in a leaflet that is included within the article of manufacture. In certain embodiments, the respiratory disorder is selected from emphysema, chronic obstructive pulmonary disease (COPD), chronic bronchitis (CB), and asthma, each of which represents a separate embodiment. George C et al. shows that increased glucose levels and systemic inflammation are associated with respiratory disorders. In certain embodiments, the subject exhibits IGt. In other embodiments, the subject exhibits T2DM. Nathan D M et al shows that reducing glucose levels prevents neuropathy. Methods for diagnosing respiratory disorders are described in George C et al. and the references cited therein.

As will be appreciated by those skilled in the art, methods for diagnosing metabolic syndrome are known. The American Heart Association guidelines state that a subject exhibiting 3 of the following traits meets the criteria for the metabolic syndrome: 1) Abdominal obesity: a waist circumference over 102 cm (40 in) in men and over 88 cm (35 inches) in women; 2) Serum triglycerides: 150 mg/dl or above, or taking medication for elevated triglycerides; 3) HDL cholesterol: 40 mg/dl or lower in men and 50 mg/dl or lower in women; 4) Blood pressure of 130/85 or above (or taking medication for high blood pressure); 5) Fasting blood glucose of 100 mg/dl or above.

In still other embodiments, there is provided a method of reducing an incidence of cardiovascular disease (CVD) in a subject with elevated glucose levels, comprising: administering to the subject a pharmaceutical composition, comprising placental ASC, thereby reducing an incidence of CVD in the aforementioned subject. In other embodiments, there is a provided a pharmaceutical composition comprising placental ASC, for reducing an incidence of CVD in the aforementioned subject. In other embodiments, there is provided use of placental ASC in the preparation of a medicament for reducing an incidence of CVD in the aforementioned subject. In other embodiments, there is provided an article of manufacture, comprising a packaging material and a pharmaceutical composition comprising placental ASC and identified for reducing an incidence of CVD in the aforementioned subject, the pharmaceutical compositions being contained within the packaging material. In some embodiments, the indication is specified in a leaflet that is included within the article of manufacture. In various embodiments, the CVD is CV mortality, non-fatal myocardial infarction [MI], non-fatal stroke, or heart failure, each of which represents a separate embodiment. In certain embodiments, the subject exhibits IGt. In other embodiments, the subject exhibits T2DM. Khaw K T et al. and Selvin E et al. show that lowering of HbA1c levels is associated with reduced CVD.

Methods for diagnosing IGt and T2DM are known in the art. The current WHO diagnostic criteria for diabetes are fasting plasma glucose ≥7.0 mmol/l (126 mg/dl) or 2-h plasma glucose ≥11.1 mmol/1 (200 mg/dl) (after ingestion of 75 g oral glucose load). The WHO criteria for Impaired Fasting Glucose (IFG) is 6.1-6.9 mmol/l. The WHO criteria for Impaired Glucose tolerance (IGt) are Fasting plasma glucose 2-h plasma glucose*<7.0 mmol/1 (126 mg/dl), and 2-h plasma glucose ≥7.8 and <11.1 mmol/1 (140 mg/dl and 200 mg/dl)

In certain embodiments, the subject with elevated blood sugar levels and/or IGt has a HbA1c value of at least 43.5 mmol/mol (corresponding to about 6.1% DCCD); is at least 60 years of age; or has a body mass index (BMI) of at least 27.5 kg/m² at the onset of treatment. In other embodiments, the patient exhibits any combination of 2 of these characteristics, or exhibits all 3 characteristics, each of which represents a separate embodiment. Alternatively or in addition, the subject exhibits Impaired Fasting Glucose (IFG).

In other embodiments, the subject has a HbA1c value in the range of 5.8%-10%, 6-10%, 6-9%, 6.5-10%, 6.5-9%, or 6-8.5% at the onset of treatment. In other embodiments, the subject has an HbA1c value of at least 5.8%.

The subject is, in still other embodiments, an adult subject. In other embodiments, the subject has an age of at least 50 years, at least 57 years, at least 58 years, at least 59 years, at least 60 years, at least 65 years, at least 70 years, or at least 75 years at the onset of treatment.

In still other embodiments, the subject is an obese subject at the onset of treatment of the present invention. In more specific embodiments, the subject has a BMI of at least 30 kg/m², at least 31 kg/m², at least 32 kg/m², at 33 kg/m², at least 34 kg/m², at least 35 kg/m², at least 36 kg/m², at least 37 kg/m², at least 38 kg/m², at least 39 kg/m² or at least 40 kg/m² at the onset of treatment. In other embodiments, there is provided a method of reducing systemic inflammation in a subject with IGt in a subject in need thereof, comprising: administering to the subject a pharmaceutical composition, comprising placental ASC, thereby reducing systemic inflammation in a subject with IGt. In other embodiments, there is a provided a pharmaceutical composition comprising placental ASC, for reducing systemic inflammation in a subject with IGt. In other embodiments, there is provided use of placental ASC in the preparation of a medicament for reducing systemic inflammation in a subject with IGt. In other embodiments, there is provided an article of manufacture, comprising a packaging material and a pharmaceutical composition comprising placental ASC and identified for reducing systemic inflammation in a subject with IGt, the pharmaceutical compositions being contained within the packaging material. In some embodiments, the indication is specified in a leaflet that is included within the article of manufacture. In certain embodiments, the systemic inflammation is secondary to IGt.

Those skilled in the art will appreciate that methods for diagnosing systemic inflammation are known in the art, and include, for example, measuring levels of C-reactive protein [CRP], interleukin-6 [IL-6], and soluble tumor necrosis factor receptor 2 [TNFR-2] (Gupta S1 et al and the references cited therein).

In certain embodiments, the subject with systemic inflammation also has a HbA1c value of at least 43.5 mmol/mol (corresponding to about 6.1% DCCD); is at least 60 years of age; or has a body mass index (BMI) of at least 27.5 kg/m² at the onset of treatment. In other embodiments, the patient exhibits any combination of 2 of these characteristics, or exhibits all 3 characteristics, each of which represents a separate embodiment. Alternatively or in addition, the subject exhibits Impaired Fasting Glucose (IFG).

In other embodiments, the subject has a HbA1c value in the range of 5.8%-10%, 6-10%, 6-9%, 6.5-10%, 6.5-9%, or 6-8.5% at the onset of treatment. In other embodiments, the subject has an HbA1c value of at least 5.8%.

The subject is, in still other embodiments, an adult subject. In other embodiments, the subject's age is at least 50 years, at least 57 years, at least 58 years, at least 59 years, at least 60 years, at least 65 years, at least 70 years, or at least 75 years at the onset of treatment.

In still other embodiments, the subject is an obese subject at the onset of any of the described treatments. In more specific embodiments, the subject's BMI is at least 30 kg/m², at least 31 kg/m², at least 32 kg/m², at 33 kg/m², at least 34 kg/m², at least 35 kg/m², at least 36 kg/m², at least 37 kg/m², at least 38 kg/m², at least 39 kg/m² or at least 40 kg/m² at the onset of treatment.

A typical dosage of the described ASC used alone ranges, in some embodiments, from about 75-500 million per dosing day. In certain embodiments, 100-400 million ASC are administered by intramuscular (IM) injection(s). In other embodiments, 100-300 million, 125-400 million, 150-400 million, 175-400 million, 200-400 million, 250-400 million, 300-400 million, 250-350 million, or 200-400 million ASC are administered by IM injection(s) per dosing day. In more specific embodiments, at least 2 doses are administered. In other embodiments, 2-10, 2-8, 2-5, 2-4, 2-3, or 2 doses are administered. In still other embodiments, at least 2 doses are administered, each dose originating from a different placental donor.

Alternatively or in addition, each dose is administered in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, 5-50, 50-60, or 50-75 injections.

In various embodiments, engraftment of the described cells in the host is not required for the cells to exert the described therapeutic effects, each of which is considered a separate embodiment. In other embodiments, engraftment is required for the cells to exert the therapeutic effect(s). For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days. In other embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Methods Utilizing Sequential ASC Doses from Different Donors

In some embodiments, the subject is administered: (a) a first pharmaceutical composition, comprising allogeneic ASC from a first donor; and subsequently (b) a second pharmaceutical composition comprising allogeneic ASC from a second donor, wherein the second donor differs from the first donor in at least one allele group of HLA-A or HLA-B; wherein the administrations are separated in time from each other by at least 7 days. Since each donor has 2 allele groups for each of HLA-A and HLA-B, the donors thus differ in at least one of the 4 allele groups.

In other embodiments, the subject is administered a pharmaceutical composition, comprising ASC, wherein the ASC have been selected from a group of populations that exhibit common characteristics but differ in their HLA types, and the subject has been tested for immunity against at least one HLA type of the selected ASC populations. In certain embodiments, the subject has been determined to lack significant immunity against said HLA type(s). HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

Those skilled in the art will appreciate that, in some embodiments, the characteristics of 2 or more ASC populations are compared by a side-by-side assay. Alternatively, the different populations can be compared in separate experiments, using side-by-side assays with the same reference standard, to which the results are normalized.

In other embodiments, the subject is administered a pharmaceutical composition, comprising ASC, wherein the ASC have been selected from a group of populations that exhibit common characteristics but differ in their HLA types. Prior to administration of the pharmaceutical composition, the subject has been administered allospecific desensitization against at least one of said HLA types. Methods for allospecific desensitization are known in the art, a non-limiting example of which is reduction of antibody titer levels of the recipient. Non-limiting examples of such methods are described, for example in Alelign T et al. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In still other embodiment, the described therapeutic method comprises the steps of: (a) testing a subject for immunity against a panel of HLA types; (b) selecting an ASC population from a group of populations from different donors, wherein the populations exhibit common characteristics but differ in their HLA types, and the subject lacks significant immunity against the HLA type of the selected population; and (c) administering a pharmaceutical composition, comprising the selected ASC population, to the subject. Thus, in some embodiments, the selected population is chosen based (at least in part) on an expected lack of significant immune reactivity of the subject for the population. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

In yet another embodiment, the described therapeutic method comprises the steps of: (a) administering a first pharmaceutical composition, comprising allogeneic ASC from a first donor, to a subject; (b) testing the subject for immunity against a panel of HLA types; (c) selecting a second ASC population from a group of populations, wherein the populations exhibit characteristics common to the ASC from the first donor, but differ in their HLA types, and the subject lacks significant immunity against the HLA type of the second ASC population; and (d) administering a second pharmaceutical composition, comprising the second ASC population, to the subject. Thus, in some embodiments, the second population is chosen based (at least in part) on an expected lack of significant immune reactivity of the subject for the population. In certain embodiments, the subject is tested for allospecific immunity after the first pharmaceutical composition is administered; while in other embodiments, the subject may be tested for allospecific immunity before the first pharmaceutical composition is administered. HLA type, in preferred embodiments, may refer to an HLA-A type. In other embodiments, HLA type refers to both HLA-A and HLA-B. In still other embodiments, HLA type refers to HLA-A, HLA-B, and HLA-C. In yet other embodiments, HLA type refers to HLA-A, HLA-B, and HLA-DR.

Significant immunity to an HLA type (allospecific immunity), as used herein, refers to a level of immunity that is expected to result in acute rejection of a tissue having the specified HLA type (Alelign T et al). Those skilled in the art will appreciate that specificity of a subject's HLA antibodies can be determined using Luminex-based assays, which may utilize, for example, fluorescent microbeads conjugated to single recombinant HLA class I and class II molecules. Such kits are commercially available, and include, for example, the One Lambda kit (ThermoFisher) and the LIFECODES LSA Single Antigen kit (Immucor).

In other embodiments, HLA antibodies present in the serum of the subject are assayed for complement-fixing ability, e.g. binding of C1q to the antibodies. Lack of complement-fixing ability above threshold levels in standard assays (Valenzuela and Reed; Chin et al.) indicates immune tolerance.

In yet other embodiments, HLAMatchmaker (Silva et al.) is used to evaluate compatibility of the subject with the described HLA populations.

In certain embodiments, the described allogeneic ASC from the first donor and the second donor (also referred to herein as “first ASC population” and “second ASC population”, respectively) exhibit common characteristics. In some embodiments, the common characteristics relate to the cells' therapeutic potential. Certain embodiments of such common characteristics are described herein. In other embodiments, the common characteristic is selected from population doubling time (PDL; this parameter may be derived from population doubling level) and glucose consumption rate (GCR), or in other embodiments is a combination thereof. In certain embodiments, the PDL and/or GCR are measured in bioreactor culture in 3D fibrous carriers, e.g. as described herein in Example 4, following cell expansion as described in Example 1. In certain embodiments, the 2 populations are within 2 fold of each other for GCR on day 5 of bioreactor culture. In other embodiments, the GCR is measured on day 3, day 4, or day 6. Alternatively or in addition, the 2 populations are within 1.5 fold, within 3 fold, or within 5 fold of each other for the specified parameter. In still other embodiments, the 2 populations secrete a therapeutic factor (which is, in some embodiments, a secreted protein) at levels within 1.5 fold, within 2 fold, within 3 fold, or within 5 fold of each other, each of which represents a separate embodiment. In certain embodiments, the therapeutic factor is any factor mentioned herein, of which represents a separate embodiment, and each of which may be freely combined with the aforementioned fold levels and other cell characteristics mentioned herein.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g. using CRISPR-mediating editing or the like). Cell populations with an intentionally modified HLA genotype are not intended to be encompassed. ASC populations that contain a mixture cells from more than one donor are also not intended to be encompassed.

As will be appreciated by those skilled in the art, the HLA system or complex is a gene complex encoding the major histocompatibility complex (MHC) proteins in humans. These cell-surface proteins are involved in regulation of the immune system in humans. The HLA gene complex resides on a 3-Mbp stretch within chromosome 6p21. HLA genes are highly polymorphic. HLAs encoding MHC class I proteins (“class I HLA's”) present peptides from inside the cell, while class II HLA's present external peptides.

There are 3 major MHC class I genes, HLA-A, HLA-B, and HLA-C; and 3 minor class I genes, HLA-E, HLA-F and HLA-G. 02-microglobulin binds with major and minor gene subunits to produce a heterodimer.

There are 3 major (DP, DQ and DR) and 2 minor (DM and DO) MHC class II proteins encoded by the HLA. The class II MHC proteins combine to form heterodimeric (αβ) protein receptors that are typically expressed on the surface of antigen-presenting cells.

HLA alleles are often named according to a multi-partite system, where the letter prefix (e.g. “HLA-A”) denotes the locus, followed by an asterisk, followed by the “allele group” number, followed by the specific HLA protein number, followed by a number used to denote silent DNA mutations in a coding region, followed by, lastly, a number used to denote DNA mutations in a non-coding region (Robinson J et al.). Typically, an allele group corresponds to a particular encoded serological antigen, while specific HLA proteins within an allele group exhibit relatively minor antigenic differences. For example, in the hypothetical allele “HLA-A*02:07:01:03”, the allele group number is 02; 07 is the specific HLA protein number; 01 describes a pattern of silent DNA mutations in the coding regions; and 03 describes a pattern of DNA mutations in non-coding regions. “Mutations” in this regard refers to variations relative to the founder (initially identified) allele in the allele group. As used herein an “antigenic” difference refers to a different allele group, while an “allelic” difference refers to a different HLA protein within the same allele group.

HLA typing at each locus, may be, in some embodiments, low resolution, or “first-level field” typing, by reference to the two digits preceding the first separator, or antigen level typing, e.g. A*02 in the above example. In various other embodiments, the typing is at “intermediate-level” resolution, i.e. second-level field, e.g. HLA-A*02:07, or in other embodiments, third-level field, e.g. HLA-A*02:07:01. In other embodiments, the typing is “allele level typing”, using all digits in the first, second, third and fourth fields, e.g. HLA-A*02:07:01:03.

Allele groups are clustered into “supertypes” which have similar peptide binding repertoires. Examples of HLA-A supertypes are 1, 2, 3, and 24, and examples of HLA-B supertypes are 7, 27, 44, 58, and 62. Typically, an allele supertype corresponds to a particular encoded serological antigen.

As provided herein (Example 5), subjects were treated with placental ASC, from 1 of 3 populations (the P041011, P090112, or P270114 population; also referred to as “04”, “09”, or “27”, respectively). Subjects treated with placental ASC from 2 different donors exhibited a superior therapeutic effect when given. In other words, serial administration of ASC from different donors is shown herein to be more efficacious than repeat administration of ASC from the same donor.

As indicated below in Table 1, 04, 09, and 27 each differ from the other batches by at least 1 of 2 alleles for HLA-A. For each combination, there is at least 1 difference in the HLA-A superfamilies, and 0 or 1 differences in the HLA-B superfamilies.

TABLE 1
HLA-A, HLA-B, HLA-C, HLA-DR, and HLA-DQ
profiles of P041011, P090112, and P270114. The
supertypes of all the HLA-A and HLA-B alleles listed
have been experimentally established (Sidney J et al).

| | P04 allele/ | P09 allele/ | P27 allele/ |
|---|---|---|---|
| Population | supertype | supertype | supertype |
| HLA-A#1 | A11:01/A03 | A11:01/A03 | A01:01/A01 |
| HLA-A#2 | A68:02/A02 | A24:02/A24 | A23:01/A24 |
| HLA-B#1 | B14:02/B27 | B44:02/B44 | B44:03/B44 |
| HLA-B#2 | B52:01/B62 | B52:01/B62 | B15:01/B62 |
| HLA-C#1 | C*08:02 | C*05:01 | C*04:01 |
| HLA-C#2 | C*12:02 | C*12:02 | C*12:03 |
| HLA-DRB1#1 | DRB1-01:02 | DRB1-12:01 | DRB1-07:01 |
| HLA-DRB1#2 | DRB1-13:02 | DRB1-15:02 | DRB1-14:01 |
| HLA-DQB1#1 | DQB1*05:01 | DQB1*03:01 | DQB1*02:02 |
| HLA-DQB1#2 | DQB1*06:09 | DQB1*06:01 | DQB1*05:03 |

Number of shared superfamilies

| | HLA-A | HLA-B |
|---|---|---|
| Combination | alleles | alleles |
| 04 vs. 09 | 1/2 | 1/2 |
| 04. vs. 27 | 0/2 | 1/2 |
| 09 vs. 27 | 1/2 | 2/2 |

Without wishing to be bound by theory, given the importance of HLA-A, HLA-B, and HLA-DR in transplant compatibility, and the lack of significant surface expression of HLA-DR in placental ASC, the present inventors propose that the additional efficacy conferred by serial treatment with P041011 and P090112 may be connected to difference(s) in their HLA-A and HLA-B alleles.

Those skilled in the art will appreciate that the protein sequences of HLA-A*11:01, HLA-A*01:01, HLA-A*68:02, HLA-A*24:02, and HLA-A*23:01 (SEQ ID Nos. 1-5, respectively) are set forth in GenBank Nucleotide Accession Nos. AY786587, EU445470, U03861, M64740, and M64742.1. The protein sequences of HLA-B*14:02, HLA-B*44:02, HLA-B*15:01, HLA-B*52:01, and HLA-B*44:03 (SEQ ID Nos. 6-10) are set forth in GenBank Nucleotide Accession Nos. M24032, M24038, U03859, M22796, and LN877362.2.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

In certain embodiments, the HLA-A alleles of the first and second donor differ from each other in at least one superfamily, while in other embodiments, they differ from each other in both superfamilies. Alternatively or additionally, the HLA-B alleles of the first and second donor differ from each other in at least one superfamily. In still other embodiments, the HLA-A alleles of the first and second donor differ from each other in at least one superfamily, while the HLA-B superfamilies do not differ. In yet other embodiments, the HLA-A alleles differ from each other in both superfamilies, while the HLA-B superfamilies do not differ.

Alternatively or in addition, the second donor differs from the first donor in at least one allele group of HLA-DR, or in other embodiments, in 2 HLA-DR allele groups.

Alternatively or in addition, the second donor differs from the first donor in at least one allele group of HLA-C, or in other embodiments, in 2 HLA-C allele groups. In still other embodiments, the second donor exhibits at least an allelic difference from the first donor in at least one allele of HLA-C, or in other embodiments, in both HLA-C alleles.

Alternatively or in addition, the second donor differs from the first donor in at least one allele group of HLA-DQ, or in other embodiments, in 2 HLA-DQ allele groups. In still other embodiments, the second donor exhibits at least an allelic difference from the first donor in at least one allele of HLA-DQ, or in other embodiments, in both HLA-DQ alleles.

In the described methods where ASC from 2 different donors are sequentially administered, step (b) (administering a second pharmaceutical composition comprising allogeneic ASC from a second donor) is, in some embodiments, performed between 2-52 weeks after step (a). In other embodiments, step (b) is performed within 4-24 weeks after step (a). In still other embodiments, step b) is performed between 3-52, 4-26, 5-26, 6-20, 6-18, 6-15, 6-10, 3-20, 3-15, 3-10, 4-12, 4-20, 5-18, 6-16, 8-16, 10-16, or 8-12 weeks after step (a).

Alternatively or in addition, step b) of the described methods is followed by an additional step, comprising the step of administering to the subject, at least 7 days after step b), a third pharmaceutical composition comprising allogeneic ASC derived from a third donor, wherein the third donor differs from both the first donor and the second donor in at least one allele group of HLA-A or HLA-B (i.e. has an allele group not represented in either the first or second donor), which is, in various embodiments, an allele of HLA-A or HLA-B. In other embodiments, the third donor differs from both the first donor and the second donor in at least two allele groups of HLA-A or HLA-B, which are, in various embodiments, an allele of HLA-A, HLA-B, or a combination thereof.

ASC and Methods of Obtaining Same

In certain embodiments, the described ASC population is plastic adherent under standard culture conditions, express the surface molecules CD105, CD73 and CD90, and do not express CD45, CD34, CD14 or CD11b, CD79α, CD19 and HLA-DR. As used herein, the phrase plastic adherent refers to cells that are capable of attaching to a plastic attachment substrate and expanding or proliferating on the substrate. In some embodiments, the cells are anchorage dependent, i.e., require attachment to a surface in order to proliferate grow in vitro.

Alternatively or in addition, the described ASC populations is placenta-derived. Except where indicated otherwise herein, the terms “placenta”, “placental tissue”, and the like refer to any portion of the placenta. Placenta-derived ASC may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis. In certain embodiments, as exemplified herein, the decidua and villi are harvested, without the amnion and chorion.

Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Placental cells may be obtained, in various embodiments, from a full-term or pre-term placenta. In some embodiments, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D substrates. Conditions for propagating adherent cells on 2D and 3D substrates are further described hereinbelow and in the Examples section which follows.

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

In still other embodiments, the described ASC population is a mixture of fetal-derived placental ASC (also referred to herein as “fetal ASC” or “fetal cells”) and maternal-derived placental ASC (also referred to herein as “maternal ASC” or “maternal cells”), where a majority of the cells are maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus).

In other embodiments, the ASC population is a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the ASC population is a placental cell population that is a mixture of fetal and maternal cells, where a majority of the cells are fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

Additional Surface Markers, Secreted Factors, and Characteristics of ASC

Alternatively or additionally, the ASC used in the described methods and compositions express a marker or a collection of markers (e.g. surface marker) characteristic of MSC or mesenchymal-like stromal cells. In some embodiments, the ASC population expresses some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the population does not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the population also lacks expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the population of ASC possesses a marker phenotype that is distinct from bone marrow-mesenchymal stem cells (BM-MSC). In certain embodiments, the ASC population is positive for expression of CD10 (which occurs, in some embodiments, in both maternal and fetal ASC); is positive for expression of CD49d (which occurs, in some embodiments, at least in maternal ASC); is positive for expression of CD54 (which occurs, in some embodiments, in both maternal and fetal ASC); is bimodal, or in other embodiments positive, for expression of CD56 (which occurs, in some embodiments, in maternal ASC); and/or is negative for expression of CD106. Except where indicated otherwise, bimodal refers to a situation where a significant percentage (e.g. at least 20%) of a population of cells express a marker of interest, and a significant percentage do not express the marker.

In certain embodiments, over 90% of the cells in the ASC population are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD73, CD29, and CD105 is expressed by more than 90% of the cells in the ASC population; and over 90% (or in other embodiments, over 95%, or in other embodiments, over 98%) of the cells in the ASC population do not differentiate into adipocytes, under conditions where mesenchymal stem cells would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 meg/ml insulin, on days 7 and 15, for a total of 25 days (“standard adipogenesis induction conditions”). In yet other embodiments, for the ASC population, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (“modified adipogenic conditions”). The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates.

“Positive” expression of a marker indicates a value higher than the range of the main peak of a fluorescence-activated cell sorting (FACS) isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

In still other embodiments, the majority, in other embodiments over 60%, over 70%, over 80%, or over 90% of the ASC in the population express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, the ASC population is less than 50%, less than 40%, less than 30%, less than 20%, or less than 10%, or less than 5% positive for CD200. In other embodiments, the ASC population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV³ NeuAc 2→3 GalGB4); Kannagi R et al), or in other embodiments both markers. Alternatively or in addition, more than 50% of the cells express HLA-A2 (UniProt Accession No. P01892). The aforementioned, non-limiting marker expression patterns were found in certain fetally-derived placental cell populations that were expanded on 3D substrates. The Uniprot entries mentioned in this paragraph were accessed on accessed on Feb. 8, 2017.

In other embodiments, each of CD29, CD73, CD90, and CD105 is expressed by more than 80% of the ASC in the population; and over 90% (or in other embodiments, over 95%, or in other embodiments, over 98%) of the cells in the population do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, and CD106 is expressed by less than 10% of the cells; less than 20% of the cells highly express CD56; and the cells do not differentiate into osteocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the aforementioned conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogeneic conditions”). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

Certain of the described maternal and fetal-derived placental cell populations are resistant to differentiation into osteocytes and/or adipocytes, are described in PCT/IB2019/054828, to Zami Aberman et al, which is incorporated herein by reference.

In other embodiments, each of CD29, CD73, CD90, and CD105 is expressed by more than 80% of the ASC population; and over 90% (or in other embodiments, over 95%, or in other embodiments, over 98%) of the cells in the population do not differentiate into adipocytes, after incubation in adipogenesis induction medium, namely a solution containing 1 mcM dexamethasone, 0.5 mM IBMX, 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD39, and CD106 is expressed by less than 10% of the cells; less than 20% of the cells highly express CD56; and the cells do not differentiate into adipocytes, after incubation under the aforementioned conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, after incubation under the aforementioned conditions; or, in other embodiments, under the modified adipogenic conditions. In still other embodiments, over 90% of the cells in the population do not differentiate into either adipocytes or osteocytes under the aforementioned standard conditions. In yet other embodiments, over 90% of the cells in the population do not differentiate into either adipocytes or osteocytes under the aforementioned modified conditions. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetally-derived placental cell populations that were expanded on 3D substrates.

Alternatively or in addition, the described ASC population exhibits immunosuppressive capability. Those skilled in the art will appreciate that immunosuppressive capability can be assayed ex vivo by mixed lymphocyte reaction (MLR). For example, human irradiated cord blood (iCB) cells are incubated with allogeneic human peripheral blood-derived monocytes (PBMC), in the presence or absence of a cell population to be tested. PBMC cell replication, which correlates with the intensity of the immune response, can be measured by ³H-thymidine uptake. Reduction of the PBMC cell replication when co-incubated with test cells indicates an immunosuppressive capability. Other exemplary methods of determining the immunosuppressive capability of a cell population are described in Example 3 of PCT Publication No. WO 2009/144720, which is incorporated herein by reference. Alternatively or in addition, secretion of pro-inflammatory and anti-inflammatory cytokines by blood cell populations (such as monocytes or PBMC) can be measured when stimulated (for example by incubation with non-matched cells, or with a non-specific stimulant such as PHA), in the presence or absence of the ASC. In certain embodiments, for example in the case of human ASC, when 200,000 PBMC are co-incubated for 48 hours with 4,000 allogeneic ASC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In other embodiments, each of CD73, CD29, and CD105 is expressed by more than 90% of the ASC population; and the ASC population inhibits proliferation of LPS-stimulated T cells. In yet other embodiments, each of CD34, CD19, and CD14 is expressed by less than 3% of the cells; and the cells inhibit T cell proliferation. In other embodiments, each of CD73, CD29, and CD105 is expressed by more than 90% of the cells, each of CD34, CD19, and CD14 is expressed by less than 3% of the cells; and the cells inhibit T cell proliferation. In certain embodiments, the inhibition of T cell proliferation, expressed as the percent decrease in T cell proliferation relative to a control, varies less than 20% (or in other embodiments, less than 10%).

In certain embodiments, the described ASC population secretes between 300-700 picograms per milliliter (pg/ml) (as exemplified herein) of VEGF per 10⁶ cells seeded, using the standard protocol (described hereinbelow). Those skilled in the art will appreciate that, since the standard protocol is performed in 2 ml medium, the number of picograms per 10⁶ cells seeded is twice the number of pg/ml, per 10⁶ cells seeded. Thus, the value of 300-700 pg/ml per 10⁶ cells seeded translates to 600-1400 pg per 10⁶ cells. Those skilled in the art will appreciate, in light of the present disclosure, that secretion levels of VEGF (or other cytokines) can be measured by methods known in the art. One possible method is seeding 1×10⁶ ASC for 20 hours in 2 mL DMEM medium; replacing the medium with EBM-2 medium, and incubating the cells under hypoxic conditions (1% 02) for an additional 24 hours; and collecting the conditioned media (CM). VEGF levels in the CM are then measured by ELISA. This is referred to herein as the “standard ELISA protocol” or “standard protocol”.

In other embodiments, the described ASC population secretes between 400-1200 pg. of PDGF-BB per 0.5×10⁶ cells seeded, using the standard protocol. In other embodiments, the population secretes at least 200, at least 300, at least 400, between 400-2,000, between 400-1500, between 500-2,000, between 500-1500, or between 500-1200 pg. of PDGF-BB per 0.5×10⁶ cells seeded, using the standard protocol.

In still other embodiments, the population secretes a factor selected from Angiogenin (Uniprot accession no. P03950), Angiopoietin 1 (Uniprot accession no. Q15389), MCP-1, IL-8, Serpin E1, and GCP2/CXCL6 (Uniprot accession no. P80162). Uniprot entries in this paragraph were accessed on Jun. 17, 2018.

In yet other embodiments, the population secretes a factor selected from IL-17, MCP-1, IL-2, CCL4/MIP-1b (Accession No. P13236), IL-4, TGF-b, TNF-alpha, IL-19, IL-20, IL-23, ADAM10-processed FasL form (sFAS; a cleavage product of TNFL6 [Accession No. P48023), Cox-2, CXCL12, CSF1, MMP-2, MMP-9, IL-32 (Accession No. P24001). Uniprot entries in this paragraph were accessed on Jun. 21, 2018.

In still other embodiments, the described ASC population secretes between 20-160 pg. (=10-80 pg/ml, as exemplified herein) of IL-6 (UniProt identifier P05231) per 10⁶ cells seeded, using the standard protocol.

In other embodiments, the described ASC population secretes or expresses (as appropriate in each case) IL-6, IL-8, eukaryotic translation elongation factor 2 (EEEF2), reticulocalbin 3, EF-hand calcium binding domain (RCN₂), and/or calponin 1 basic smooth muscle (CNN1), when tested using the aforementioned standard protocol.

Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% fetal bovine serum (FBS)+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

In certain embodiments, the described ASC population stimulates endothelial cell proliferation (ECP). Those skilled in the art will appreciate that ECP activity can be assayed ex vivo by seeding 1×10⁶ ASC in 2 mL DMEM medium, in wells of a 6-well plate for 20 hours, then replacing the medium with EBM-2 medium (available from Sigma-Aldrich) and incubating the cells under hypoxic conditions (1% 02) for an additional 24 hours. Afterwards, the conditioned media (ASC-CM) is collected. 750 Human Umbilical Vein Endothelial Cells (HUVECs) cells are seeded per well of 96-well plate were seeded and incubated for 24 hours in EBM-2 medium and then incubated with the ASC-CM, for 4 days under normoxic conditions (21% O₂) at 37° C., and proliferation is assayed.

In other embodiments, the described ASC population secretes a therapeutic moiety, which is, in some embodiments, a secreted protein. In still other embodiments, the therapeutic moiety is VEGF. Those skilled in the art will appreciate, in light of the present disclosure, that secretion levels of VEGF can be measured by methods known in the art, e.g. the described standard ELISA protocol.

Alternatively or in addition, the described ASC population secretes between 600-2000 pg. (=300-1000 pg/ml, as exemplified herein) of VEGF per 10⁶ cells seeded, using the standard protocol. In other embodiments, the population secretes at least 400, at least 600, at least 800, between 600-1600, between 600-1400, between 600-1200, between 800-2000, between 800-1600, between 800-1400, or between 800-1200 pg. of VEGF per 10⁶ cells seeded, using the standard protocol described herein.

In yet other embodiments, the therapeutic moiety is Angiogenin. Those skilled in the art will appreciate, in light of the present disclosure, that secretion levels of Angiogenin can be measured by methods known in the art, e.g. the described standard ELISA protocol. Alternatively or in addition, the described ASC population secretes between 400-800 pg. (=200-400 pg/ml, as exemplified herein) of Angiogenin per 10⁶ cells seeded, using the standard protocol.

In yet other embodiments, the therapeutic moiety is Serpin E1. Those skilled in the art will appreciate, in light of the present disclosure, that secretion levels of Serpin E1 can be measured by methods known in the art, e.g. the described standard ELISA protocol. Alternatively or in addition, the described ASC population secretes between 30,000-60,000 pg. (=15,000-30,000 pg/ml, as exemplified herein) of Serpin E1 per 10⁶ cells seeded, using the standard protocol.

In yet other embodiments, the therapeutic moiety is MMP-1. Those skilled in the art will appreciate, in light of the present disclosure, that secretion levels of MMP-1 can be measured by methods known in the art, e.g. the described standard ELISA protocol. Alternatively or in addition, the described ASC population secretes between 8000-400,000 pg. (=4000-200,000 pg/ml, as exemplified herein) of MMP-1 per 10⁶ cells seeded, using the standard protocol.

In still other embodiments, the described ASC population secretes Flt-3 ligand (Fms-related tyrosine kinase 3 ligand; Uniprot Accession No. P49772), stem cell factor (SCF; Uniprot Accession No. P21583), IL-6, or combinations thereof, each of which represents a separate embodiment. Uniprot entries in this and the following 2 paragraphs were accessed on Feb. 26, 2017.

In other embodiments, the described ASC population secretes 2 or more, in other embodiments 3 or more, in other embodiments 4 or more, in other embodiments 5 or more, in other embodiments 6 or more, in other embodiments 7 or more, or in other embodiments all of the factors VEGF, Angiogenin, PDGF, Angiopoietin 1, MCP-1, IL-8, Serpin E1, and GCP2/CXCL6. In other embodiments, the ASC secrete VEGF, Angiogenin, Angiopoietin 1, MCP-1, IL-8, and Serpin E1, which were found to be secreted by maternal cells. In still other embodiments, the ASC secrete VEGF, Angiogenin, Angiopoietin 1, MCP-1, IL-8, Serpin E1, and GCP2/CXCL6, which were found to be secreted by fetal cells.

In still other embodiments, the described ASC population secretes 2 or more, in other embodiments 3 or more, in other embodiments 4 or more, in other embodiments 5 or more, in other embodiments 6 or more, or in other embodiments 7 or more factors selected from MCP-1 (CCL2), Osteoprotegerin, MIF (Macrophage migration inhibitory factor; Uniprot Accession No. P14174), GDF-15, SDF-1 alpha, GROa (Growth-regulated alpha protein/CXCL1; Uniprot Accession No. P09341), Beta2-Microglobulin (beta2M; this protein, although it forms complexes with the heavy chain of MHC class I, can also be secreted [Nomura T et al]), IL-6, IL-8 (UniProt identifier P10145), ENA78/CXCL5, eotaxin/CCL11 (Uniprot Accession No. P51671), and MCP-3 (CCL7). In certain embodiments, the ASC secrete MCP-1, Osteoprotegerin, MIF, GDF-15, SDF-1 alpha, GROa, beta2M, IL-6, IL-8, and MCP-3, which were found to be secreted by maternal cells. In other embodiments, the ASC secrete MCP-1, Osteoprotegerin, MIF, GDF-15, SDF-1 alpha, beta2M, IL-6, IL-8, ENA78, eotaxin, and MCP-3, which were found to be secreted by fetal cells. All Swissprot and UniProt entries in this paragraph were accessed on Mar. 23, 2017.

In yet other embodiments, the described ASC population secretes IGFBP-1 (Insulin-like growth factor-binding protein 1; Accession No. P08833). Those skilled in the art will appreciate, in light of the present disclosure, that secretion levels of IGFBP-1 can be measured by methods known in the art, e.g. the described standard ELISA protocol. Alternatively or in addition, the described ASC population secretes between 220-500 pg. (=110-250 pg/ml, as exemplified herein) of IGFBP-1 per 10⁶ cells seeded, using the standard protocol.

In yet other embodiments, the described ASC population secretes IGFBP-3. Those skilled in the art will appreciate, in light of the present disclosure, that secretion levels of IGFBP-3 can be measured by methods known in the art, e.g. the described standard ELISA protocol. Alternatively or in addition, the described ASC population secretes between 2,000-14,000 pg. (=1000-7000 pg/ml; as exemplified herein) of IGFBP-3 per 10⁶ cells seeded, using the standard protocol.

In other embodiments, the therapeutic moiety is selected from bFGF (basic fibroblast growth factor; accession no. P09038), NGF (nerve growth factor; Accession No. P01138), VEGF, LIF (Leukemia inhibitory factor; Accession No. P15018), MIF, MCP-1, PDGF (a non-limiting example of which is PDGF-AA), Angiogenin, IGFBP-3, and G-CSF. In yet other embodiments, the factor is selected from M-CSF, SDF-1, IFN-g, MMP-1, BMP-4 (Bone morphogenetic protein 4; Accession No. P12644), HB-EGF (Proheparin-binding EGF-like growth factor; Accession No. Q99075), GM-CSF, and ENA78. Uniprot Accession Nos. in this paragraph were accessed on Jun. 20, 2018.

Alternatively or in addition, the ASC secrete immunoregulatory and/or anti-inflammatory factor(s), which may be, in some embodiments, any factor described herein. Other relevant embodiments are described in WO/2007/108003, which is incorporated herein by reference.

In other embodiments, the described ASC population secretes increases secretion of IL-10 (Uniprot Accession No. P22301; record accessed on Jun. 17, 2018) by allogeneic monocytes over basal secretion, when the ASC are cocultured with the monocytes. Those skilled in the art will appreciate in light of the present disclosure that IL-10 secretion can be assayed by seeding 3000 ASC/well in X-VIVO™ 15 medium+10% FBS in 48-well plates and, 1 day later, co-incubating the ASC with 2×10⁴ U937 cells and incubating for 17 hours. PHA is then added, cells are incubated for another 5 hours, and IL-10 in the supernatant is measured by ELISA. In certain embodiments, the amount of IL-10 secretion by the monocytes is at least 120%, at least 130%, at least 150%, at least 170%, at least 200%, or at least 300% of the amount observed with LPS stimulation in the absence of ASC.

In other embodiments, the described ASC population secretes an immunoregulatory factor(s). In some embodiments, the therapeutic moiety is Leukemia Inhibitory Factor (LIF). Those skilled in the art will appreciate, in light of the present disclosure, that secretion levels of LIF can be measured by methods known in the art.

In still other embodiments, the therapeutic moiety is GROa (Growth-regulated alpha protein). Those skilled in the art will appreciate, in light of the present disclosure, that secretion levels of GROa can be measured by methods known in the art, e.g. the described standard ELISA protocol.

Alternatively or in addition, the described ASC population secretes between 40-200 pg. (=100 pg/ml, as exemplified herein) of GROa per 10⁶ cells seeded, using the standard protocol.

In still other embodiments, the therapeutic moiety is IL-8. Those skilled in the art will appreciate, in light of the present disclosure, that secretion levels of IL-8 can be measured by methods known in the art, e.g. the described standard ELISA protocol. Alternatively or in addition, the described ASC population secretes between 100-700 pg. (=50-350 pg/ml, as exemplified herein) of IL-8 per 10⁶ cells seeded, using the standard protocol.

In still other embodiments, the therapeutic moiety is SDF-1/CXCL12 (Uniprot Accession No. P48061) (SDF-1 alpha, assayed herein, is a cleavage product of SDF-1). Those skilled in the art will appreciate, in light of the present disclosure, that secretion levels of SDF-1 can be measured by methods known in the art, e.g. the described standard ELISA protocol. Alternatively or in addition, the described ASC population secretes between 150-500 pg. (=125-250 pg/ml, as exemplified herein) of SDF-1 per 10⁶ cells seeded, using the standard protocol.

In still other embodiments, the therapeutic moiety is Osteoprotegerin (Uniprot Accession No. 000300). Those skilled in the art will appreciate, in light of the present disclosure, that secretion levels of Osteoprotegerin can be measured by methods known in the art, e.g. the described standard ELISA protocol. Alternatively or in addition, the ASC population secretes between 200-1400 (which equals 100-700 pg/ml) of Osteoprotegerin per 10⁶ cells seeded, using the standard protocol.

In still other embodiments, the therapeutic moiety is MIF (Macrophage migration inhibitory factor). Those skilled in the art will appreciate, in light of the present disclosure, that secretion levels of MIF can be measured by methods known in the art, e.g. the described standard ELISA protocol. Alternatively or in addition, the described ASC population secretes between 2000-8000 pg. (=1000-4000 pg/ml, as exemplified herein) of MIF per 10⁶ cells seeded, using the standard protocol.

In yet other embodiments, the therapeutic moiety is M-CSF (Accession No. P09603). Those skilled in the art will appreciate, in light of the present disclosure, that secretion levels of M-CSF can be measured by methods known in the art, e.g. the described standard ELISA protocol. Alternatively or in addition, the described ASC population secretes (as exemplified herein) between 300-800 pg. of M-CSF per 0.5×10⁶ cells seeded, using the standard protocol.

In other embodiments, the therapeutic moiety is selected from MCP-1 (CCL2), GDF-15, IL-6, IL-8, ENA78/CXCL5, eotaxin/CCL11, MCP-3 (CCL7), GM-CSF, HGF, G-CSF, IL-10, CCL5 (RANTES; Accession No. P13501), sICAM-1 (Accession No. Q99930), Osteopontin, TGF-01, IL-11, IDO (Indoleamine 2,3-dioxygenase 1; Accession No. P14902) and PD-L1 (CD274; Accession No. Q9NZQ7). In still other embodiments, the therapeutic moiety is a hormone, a non-limiting example of which is PGE2 (ChEMBL identifier CHEMBL548). Uniprot Accession Nos. in this paragraph were accessed on Jun. 18, 2018.

Alternatively or in addition, the ASC secrete a factor(s) selected from G-CSF (Granulocyte colony-stimulating factor; Uniprot Accession No. P09919); GM-CSF (Granulocyte-macrophage colony-stimulating factor; Uniprot Accession No. P04141); GROa/CXCL1; IL-6; IL-8; MCP-1, MCP-3 (Monocyte chemoattractant proteins 1 and 3/UniProt Nos. P13500 and P80098, respectively), ENA78 (CXCL5; Uniprot Accession No. P42830); LIF (Leukemia inhibitory factor); EPO (Erythropoietin; UniProt identifier P01588), IL-3 (interleukin-3; Uniprot Accession No. P08700), and SCF. Other relevant embodiments are described in PCT/IB2018/051601, which is incorporated herein by reference.

In other embodiments, the cells in the ASC population exhibit a spindle shape when cultured under 2D conditions.

According to some embodiments, the ASC population expresses CD200, while in other embodiments, the population lacks expression of CD200. In still other embodiments, less than 30%, 25%, 20%, 15%, 10%, 8%, 6%, 5%, 4%, 3%, or 2%, 1%, or 0.5% of the adherent cells express CD200. In yet other embodiments, greater than 70%, 75%, 80%, 85%, 90%, 92%, 94%, 95%, 96%, 97%, 98%, 99%, or 99.5% of the adherent cells express CD200.

In still other embodiments, the cells in the ASC population are allogeneic, or in other embodiments, the cells are autologous. In other embodiments, the cells are fresh or, in other embodiments, frozen (for example, cryopreserved).

In various embodiments, any of the embodiments of surface marker expression and other characteristics may be freely combined with the described embodiments of cytokine expression.

Use of Serum-Deficient Medium and Serum-Replacement Medium

In other embodiments, the described cell population is produced by expanding a cell population (for example, a population of placental adherent cells) in a medium that contains less than 5% animal serum. In certain embodiments, the aforementioned medium contains less than 4% animal serum; less than 3% animal serum; less than 2% animal serum; less than 1% animal serum; less than 0.5% animal serum; less than 0.3% animal serum; less than 0.2% animal serum; or less than 0.1% animal serum. In other embodiments, the medium does not contain animal serum, which may be referred to as “serum-replacement medium”. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In certain embodiments, the serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

In other embodiments, the serum-deficient medium contains one or more growth factors. In certain embodiments, the growth factors, individually or, in other embodiments collectively, induce cell expansion in culture. In other embodiments, the growth factors, individually or, in other embodiments collectively, induce cell expansion in culture without differentiation.

In more specific embodiments, the factor(s) contained in the serum-deficient medium is selected from a FGF, TGF-beta (Uniprot accession no. P01137), transferrin (e.g. serotransferrin or lactotransferrin; Uniprot accession nos. P02787 and P02788), insulin (Uniprot accession no. P01308), EGF (epidermal growth factor; Uniprot accession no. P01133), and/or PDGF (platelet-derived growth factor, including any combination of subunits A and B; Uniprot accession nos. P04085 and P01127), each of which represents a separate embodiment. A non-limiting example of PDGF is PDGF-BB.

Except where indicated otherwise, reference herein to a protein includes all its isoforms functional fragments thereof, and mimetics thereof. Such reference also includes homologues from a variety of species, provided that the protein acts on the target cells in a similar fashion to the homologue from the same species as the target cells. For example, if human cells are being expanded, reference to bFGF would also include any non-human bFGF that stimulates proliferation of human cells. Those skilled in the art will appreciate that, even in the case of human cells, the aforementioned proteins need not be human proteins, since many non-human (e.g. animal) proteins are active on human cells. Similarly, the use of modified proteins that have similar activity to the native forms falls within the scope of the described methods and compositions.

Other examples of serum-deficient and serum-replacement medium are described in WO 2019/186471, to Lior Raviv et al., which is hereby incorporated by reference.

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 mcg/ml) and oleic acid are present; or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

Incubation with Pro-Inflammatory Cytokines

In certain embodiments, the described ASC population has been incubated with pro-inflammatory cytokines. Reference herein to one or more “pro-inflammatory” cytokines, or “inflammatory cytokines”, which is used interchangeably, implies the presence of at least one cytokine that mediates an inflammatory response in a mammalian host, for example a human host. A non-limiting list of cytokines are Interferon-gamma (IFN-gamma; UniProt identifier P01579), IL-22 (UniProt identifier Q9GZX6), Tumor Necrosis Factor-alpha (TNF-alpha; UniProt identifier P01375), IFN-alpha, IFN-beta (UniProt identifier P01574), IL-1alpha (UniProt identifier P01583), IL-1beta (UniProt identifier P01584), IL-17 (UniProt identifier Q5QEX9), IL-23 (UniProt identifier Q9NPF7), IL-17A (UniProt identifier Q16552), IL-17F (UniProt identifier Q96PD4), IL-21 (UniProt identifier Q9HBE4), IL-13 (UniProt identifier P35225), IL-5 (UniProt identifier P05113), IL-4 (UniProt identifier P05112), IL-33 (UniProt identifier 095760), IL-1RL1 (UniProt identifier Q01638), TNF-βeta (UniProt identifier P01374), IL-11 (UniProt identifier P20809), IL-9 (UniProt identifier P15248), IL-2 (UniProt identifier P60568), Tumor Necrosis Factor-Like Ligand (TL1A; a.k.a. TNF ligand superfamily member 15; UniProt identifier 095150), IL-12 (UniProt identifiers P29459 and P29460 for the alpha- and beta subunits, respectively), and IL-18 (UniProt identifier Q14116). Additional cytokines include (but are not limited to): Leukemia inhibitory factor (LIF), oncostatin M (OSM; UniProt identifier P13725), ciliary neurotrophic factor (CNTF (UniProt identifier P26441), and IL-8 (UniProt identifier P10145). All Swissprot and UniProt entries in this application were accessed on Jul. 24, 2014, except where indicated otherwise.

Except where indicated otherwise, reference to a cytokine or other protein is intended to include all isoforms of the protein. For example, IFN-alpha includes all the subtypes and isoforms thereof, such as but not limited to IFN-alpha 17, IFN-alpha 4, IFN-alpha 7, IFN-alpha 8, and IFN-alpha 110. Some representative UniProt identifiers for IFN-alpha are P01571, P05014, P01567, P32881, and P01566. Those skilled in the art will appreciate that, even in the case of human cells, the aforementioned cytokines need not be human cytokines, since many non-human (e.g. animal) cytokines are active on human cells. Similarly, the use of modified cytokines that have similar activity to the native forms falls within the scope of the described embodiments.

In certain embodiments, one or more of the cytokines is TNF-alpha. In more specific embodiments, the TNF-alpha may be the only cytokine present, or, in other embodiments, may be present together with 1, 2, 3, 4, 5, 6, 1-2, 1-3, 1-4, 1-5, or 1-6, or more than 6 added inflammatory cytokines.

In some embodiments, TNF-alpha is present together with IFN-gamma. These two cytokines may be the only 2 added cytokines, or, in other embodiments, present with additional proinflammatory cytokines.

In certain embodiments, one or more of the cytokines is IFN-gamma. In more specific embodiments, the IFN-gamma may be the only cytokine present, or, in other embodiments, may be present together with 1, 2, 3, 4, 5, 6, 1-2, 1-3, 1-4, 1-5, or 1-6, or more than 6 added cytokines.

Other embodiments of incubation of ASC with pro-inflammatory cytokines are described in PCT Publ. No. WO2017/212309, to Eytan Abraham et al, which is incorporated herein by reference.

Additional Aspects of Methods for Expansion and Preparation of ASC

In certain embodiments, the described ASC population has been subject to a 3D incubation, as described further herein. In more specific embodiments, the ASC have been incubated in a 2D adherent-cell culture apparatus, prior to the step of 3D culturing. In some embodiments, cells (which have been extracted, in some embodiments, from placenta, from adipose tissue, etc.) are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer. An apparatus suitable for such are is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of ASC are described in PCT Application Publ. No. WO/2007/108003, which is fully incorporated herein by reference in its entirety.

In various embodiments, “an adherent material” refers to a material that is suitable for cell attachment thereto. In certain embodiments, the material is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of ASC under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Other bioreactor culturing embodiments are described in WO 2019/186471, to Lior Raviv et al., which is hereby incorporated by reference.

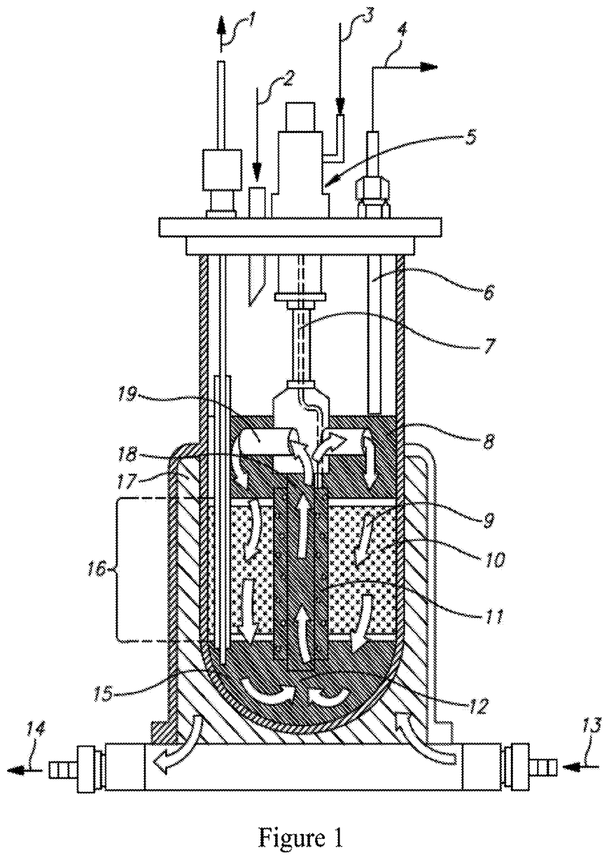

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stirring rate is used to promote cell attachment, then the stirring rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or both together. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is suitable for cell attachment thereto.

In still other embodiments, the matrix is similar to the Celligen™ Plug Flow bioreactor which is, in certain embodiments, packed with Fibra-cel® carriers (or, in other embodiments, other carriers). The spinner is, in certain embodiments, batch fed (or in other alternative embodiments fed by perfusion), fitted with one or more sterilizing filters, and placed in a tissue culture incubator.

In further embodiments, cells are seeded onto the scaffold by suspending them in medium and introducing the medium to the apparatus.

In certain embodiments, the bioreactor is seeded at a concentration of between 10,000-2,000,000 cells/ml of medium.

In still other embodiments, between 1-20×10⁶ cells per gram (gr) of carrier (substrate) are seeded, or in other embodiments 1.5-20×10⁶ cells/gr carrier, or in other embodiments 1.5-18×10⁶, or in other embodiments 1.8-18×10⁶, or in other embodiments 2-18×10⁶, or in other embodiments 3-18×10⁶, or in other embodiments 2.5-15×10⁶, or in other embodiments 3-15×10⁶, or in other embodiments 3-14×10⁶, or in other embodiments 3-12×10⁶, or in other embodiments 3.5-12×10⁶, or in other embodiments 3-10×10⁶, or in other embodiments 3-9×10⁶, or in other embodiments 4-9×10⁶, or in other embodiments 4-8×10⁶, or in other embodiments 4-7×10⁶, or in other embodiments 4.5-6.5×10⁶ cells/gr carrier.

In other embodiments, over 5×10⁵, over 7×10⁵, over 8×10⁵, over 9×10⁵, over 10⁶, over 1.5×10⁶, over 2×10⁶, over 3×10⁶, over 4×10⁶, or over 5×10⁶ viable cells are removed per milliliter of the growth medium in the bioreactor. In still other embodiments over between 5×10⁵-1.5×10⁷, between 7×10⁵-1.5×10⁷, between 8×10⁵-1.5×10⁷, between 1×10⁶-1.5×10⁷, between 5×10⁵-1×10⁷, between 7×10⁵-1×10⁷, between 8×10⁵-1×10⁷, between 1×10⁶-1×10⁷, between 1.2×10⁶-1×10⁷, or between 2×10⁶-1×10⁷ viable cells are removed per milliliter of the growth medium in the bioreactor.

In other embodiments, incubation of ASC may comprise microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.), Superbeads (commercially available from Flow Labs, Inc.), and DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

In certain embodiments, further steps of purification or enrichment for ASC may be performed. Such methods include, but are not limited to, cell sorting using markers for ASC. Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, at least 12%, at least 14%, at least 16%, at least 18%, at least 20%, at least 22%, at least 24%, at least 26%, at least 28%, or at least 30% of the cells are in the S and G2/M phases (collectively), at the time of harvest from the bioreactor. Cell cycle phases can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells.

In certain embodiments, the harvesting process comprises vibration or agitation, for example as described in PCT International Application Publ. No. WO 2012/140519, which is incorporated herein by reference. Other harvesting embodiments are described in WO 2019/186471, to Lior Raviv et al., which is hereby incorporated by reference.

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

Exosomes

In yet other embodiments, extracellular vesicles, e.g. exosomes, secreted by the described ASC are used in the described methods and compositions. Methods of isolating exosomes are well known in the art, and include, for example, immuno-magnetic isolation, for example as described in Clayton A et al., 2001; Mathias R A et al., 2009; and Crescitelli R et al., 2013.

Exosomes are, in other embodiments, identified based on their size, e.g. 40-100 nm, and/or their particular cup shape (Nilsson J et al.). In still other embodiments, exosomes are identified by glycoaffinity capture (Palmissano et al.).

In certain embodiments, the described methods comprise isolation of exosomes, for example as described in Conde-Vancells et al. and Koga et al., or the references cited therein. One such protocol, provided solely for purposes of exemplification, involved centrifuging samples for 30 min at 1500×g to remove large cellular debris. The resultant supernatants are subjected to filtration on 0.22 μm pore filters, followed by ultra-centrifugation at 10 000×g and 100 000×g for 30 and 60 min, respectively. The resulting pellets are suspended in PBS, pooled, and again ultracentrifuged at 100 000×g for 60 min. The final pellet (containing vesicles) is suspended in 150 μL of PBS, aliquoted and stored at −80° C. For higher-purity preparations, exosomes can be further purified on sucrose-containing gradients (e.g. a 30% sucrose cushion), e.g. as described in Théry C et al. Vesicle preparations are diluted in PBS and under-layered on top of a density cushion composed of pH-buffered 30% sucrose (optionally containing deuterium oxide (D₂O)), around pH 7.4, forming a visible interphase. The samples are ultracentrifuged at 100 000×g at 4° C. for 75 min in a swinging bucket rotor, and the gradient is withdrawn in aliquots from the bottom. Vesicles contained in the 30% sucrose/D₂O cushion are collected, diluted in buffered solution, and optionally centrifuged at 100 000×g to concentrate the contents. Kits for exosome isolation are available commercially, non-limiting examples of which are ExoQuick® reagents, ExoMAX Opti enhancer, and ExoFLOW products, all of which can be obtained from System Biosciences (Palo Alto, Calif.).

In some embodiments, the exosomes or other extracellular vesicles are harvested from a 3D bioreactor in which the ASC have been incubated. Alternatively or in addition, the cells are cryopreserved, and then are thawed, after which the exosomes are isolated. In some embodiments, after thawing, the cells are cultured in 2D culture, from which the exosomes are harvested.

Pharmaceutical Compositions

The described ASC can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise ASC in combination with an excipient, e.g., a pharmacologically acceptable excipient. In further embodiments, the excipient is an osmoprotectant or cryoprotectant, an agent that protects cells from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. In more specific embodiments, DMSO may be present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin. The cells may be any embodiment of ASC mentioned herein, each of which is considered a separate embodiment.

Provided in addition are pharmaceutical compositions, comprising the described placental ASC, in the absence of non-placental cell types.

Since non-autologous cells may in some cases induce an immune reaction when administered to a subject, several approaches may be utilized according to the methods provided herein to reduce the likelihood of rejection of non-autologous cells. In some embodiments, these approaches include suppressing the recipient immune system. In some embodiments, this may be done regardless of whether the ASC themselves engraft in the host. For example, the majority of the cells may, in various embodiments, not survive after engraftment for more than 3 days, more than 4 days, more than 5 days, more than 6 days, more than 7 days, more than 8 days, more than 9 days, more than 10 days, or more than 14 days.

Examples of immunosuppressive agents that may be used in the methods and compositions provided herein include, but are not limited to, methotrexate, cyclophosphamide, cyclosporine, cyclosporine A, chloroquine, hydroxychloroquine, sulfasalazine (sulphasalazopyrine), gold salts, D-penicillamine, leflunomide, azathioprine, anakinra, infliximab (REMICADE), etanercept, TNF-alpha blockers, biological agents that antagonize one or more inflammatory cytokines, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors, and tramadol.

One may, in various embodiments, administer the pharmaceutical composition in a systemic manner (as detailed hereinabove). Alternatively, one may administer the pharmaceutical composition locally, for example, via injection of the pharmaceutical composition directly into an exposed or affected tissue region of a patient. In other embodiments, the cells are administered intravenously (IV), subcutaneously (SC), by the intraosseous route (e.g. by intraosseous infusion), or intraperitoneally (IP), each of which is considered a separate embodiment. In other embodiments, the ASC or composition is administered intramuscularly; or, in other embodiments, systemically. In this regard, “intramuscular” administration refers to administration into the muscle tissue of a subject; “subcutaneous” to administration just below the skin; “intravenous” to administration into a vein of a subject; “intraosseous” to administration directly into bone marrow; and “intraperitoneal” refers to administration into the peritoneum of a subject. In still other embodiments, the cells are administered intratracheally, intrathecally, by inhalation, or intranasally. In certain embodiments, lung-targeting routes of administration may utilize cells encapsulated in liposomes or other barriers to reduce entrapment within the lungs.

In other embodiments, for injection, the described cells may be formulated in aqueous solutions, e.g. in physiologically compatible buffers such as Hank's solution, Ringer's solution, or physiological salt buffer, optionally in combination with medium containing cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described ASC are suitably formulated as a pharmaceutical composition which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described ASC may be freely combined with each embodiment relating to a therapeutic method or pharmaceutical composition.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternally-derived cells were cultured in 2D culture, followed by bioreactor culture on fibrous carriers, as described in Example 1 of International Patent Application WO 2016/098061, in the name of Esther Lukasiewicz Hagai et al, published on Jun. 23, 2016, which is incorporated herein by reference in its entirety.

To detach the cells from the carriers, carriers were incubated with trypsin solution for 4 minutes, with oscillating mixing at 5 Hz, as described in PCT International Application Publ. No.

WO 2012/140519. The medium was drained into a harvest bag, containing FBS (final concentration 10%), and the carriers were washed with isotonic solution, with oscillating mixing at 5-Hz frequency, and the cell suspension was drained into the harvest bag.

Afterwards, cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 2: Intermediate Cell Stock Production in Serum-Free

Medium

Methods

The procedure included periodic testing of the medium for sterility and contamination.

### Step 1-1—Extraction and Plating of Adherent Stromal Cells (ASC's)

Placentas were obtained from donors up to 35 years old, who were pre-screened and determined to be negative for hepatitis B, hepatitis C, HIV-1 and HIV-2, HTLV-1 and HTLV-2, and syphilis. The donor placenta was maintained sterile and cooled.

Within 36 hours of the delivery, the placenta (including the decidua and villi, and excluding the amnion and chorion) was placed with the maternal side facing upwards and minced. Pieces were washed with isotonic buffer+gentamicin, then incubated for 1-3 hours with collagenase and DNAse in isotonic buffer. DMEM with 10% filtered FBS, L-Glutamine, and gentamicin was added, and cells were filtered through a sterile stainless steel sieve and centrifuged. The cells were suspended in culture medium, seeded in flasks, and incubated at 37° C. in a humidified tissue culture incubator with 5% CO₂.

After 2 days, cells were washed with PBS, and CellStart™ cell attachment solution and StemPro® MSC SFM XenoFree medium (serum-free and xeno-free culture medium [SFM-XF]) (ThermoFisher Scientific, catalog no. A10675-01; hereinafter “StemPro® medium”) were added.

### Step 1-2—Initial Culturing

Cells were cultured for 2 additional passages (typically 4-10 population doublings after the first passage) in StemPro® medium+CellStart™. When reaching 60-90% confluence, cells were detached using trypsin, centrifuged, and seeded at 3.16±0.5×10³ cells/cm² in tissue culture flasks.

### Step 1-3—Cell Concentration, Washing, Formulation, Filling and Cryopreservation

The cell suspension from the final passage was centrifuged and suspended in culture medium at 20-40×10⁶ cells/milliliter (mL), then adjusted to 10% DMSO, 40% FBS, and 50% DMEM, the temperature was reduced in a controlled rate freezer, and cells were stored in a liquid nitrogen freezer to produce the ICS.

Results

Cell characteristics of several batches were assessed (Table 2).

TABLE 2
Characteristics of placental cells expanded in SF
medium. PDL refers to population doubling level—
in this case, the number of doublings since passage 1.

| | | | Total | cell | |
|---|---|---|---|---|---|
| | | | growth | size | |
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 20 | 18.9 | 6.9 |
| PD240214SFM | A | 1 | 7 | 16.2 | NA |
| | | 2 | 14 | 20.8 | 2.7 |
| | | 3 | 20 | 19.4 | 6.4 |
| | B | 1 | 7 | 22 | NA |
| | | 2 | 14 | 18.2 | 2.1 |
| | | 3 | 20 | 19.2 | 6.1 |
| PD230414SFM | NA | 1 | 7 | NA | NA |
| | | 2 | 14 | NA | 2.3 |
| | | 3 | 19 | 16.2 | 5.7 |
| PD040514SFM | NA | 1 | 7 | NA | NA |
| | | 2 | 14 | NA | 2.7 |
| | | 3 | 18 | 15.6 | 6.5 |
| PD260514SFM | NA | 1 | 7 | NA | NA |
| | | 2 | 13 | NA | 2.9 |
| | | 3 | 17 | 15.8 | 6.6 |
| PD180814SFM | NA | 1 | 6 | NA | NA |
| | | 2 | 10 | NA | 2.1 |
| | | 3 | 16 | 16.7 | 5.3 |
| PD220914SFM | unfiltered | 1 | 8 | NA | NA |
| | | 2 | 14 | NA | 2.1 |
| | | 3 | 20 | 17 | 5.6 |
| | filtered | 1 | 8 | NA | NA |
| | | 2 | 14 | NA | 2 |
| | | 3 | 20 | 17.8 | 5.1 |
| PD271014SFM | filtered | 1 | 9 | NA | NA |
| | | 2 | 15 | NA | 2.1 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Additional Culturing Steps

### Step 2-1: Additional Two-Dimensional (2D) Cell Culturing.

The ICS was thawed, diluted with and cultured in StemPro® medium until 60-90% confluence (typically 4-7 days after seeding), and cultured for 2 additional passages (referred to as passages 3/1 and 3/2 respectively; again passaging when reaching 60-90% confluence), then were harvested for seeding in the bioreactor.

### Step 2-2: Three Dimensional (3D) Cell Growth in Bioreactor/s

Each bioreactor contained Fibra-cel© carriers (New Brunswick Scientific) made of polyester and polypropylene, and StemPro® medium.

The culture medium in the bioreactor/s was kept at the following conditions: temp: 37±1° C., Dissolved Oxygen (DO): 70±20% and pH 7.4±0.4. Filtered gases (Air, CO₂, N₂ and O₂) were supplied as determined by the control system in order to maintain the target DO and pH values.

After seeding, the medium was stirred with stepwise increases in the speed, up to 150-200 RPM by 24 hours. Perfusion was initiated several hours after seeding and was adjusted on a daily basis in order to keep the glucose concentration constant at approximately 550 mg†liter.

Cells were typically harvested after 5-6 days by washing the cells, adding trypsin, and subjecting them to agitation.

### Step 2-3: Downstream Steps: Concentration, Washing, Formulation, and Cryopreservation

Cells were suspended and washed in suspension solution (5% w/v human serum albumin [HSA] in isotonic solution), then adjusted to 10-20×10⁶ cells/ml, in isotonic solution with 10% DMSO v/v and 5% HSA w/v. The vials were gradually chilled and stored in a gas-phase liquid nitrogen freezer.

### Example 4: Production of ASC with Different HLA Types and Similar

Therapeutic Characteristics

Methods

Endothelial cell proliferation (ECP), Monocyte IL-10 secretion, Luminex® assays, RayBiotech cytokine array, ELISAs, and PBMC proliferation inhibition assay were performed as described in PCT/IB2019/054828, to Zami Aberman et al, which is incorporated herein by reference.

Results

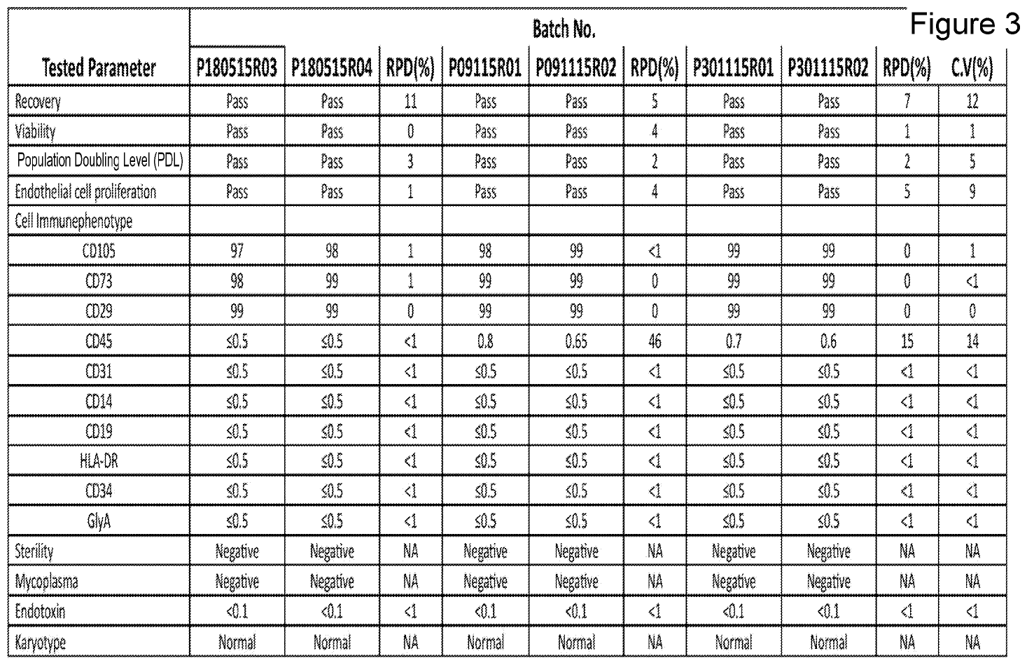

ASC were prepared from various donor placentas and subjected to 2D, followed by 3D culture, as described in Example 1. The cells removed from the 3D carriers exhibited a high degree of consistency in various characteristics, including immuno-phenotype, karyotype, population doubling level (PDL) and ECP activity (FIG. 3), and similar GCR (Table 3), while having different HLA types.

TABLE 3
GCR of different placental ASC batches in mg/day.

| Batch | Parameter | Day 3 | Day 4 | Day 5 | Day 6 |
|---|---|---|---|---|---|
| 04 | Average | 2149 | 4444 | 7624 | 11593 |
| | SE | 83 | 211 | 702 | 318 |
| 09 | Average | 3024 | 5888 | 9927 | 13953 |
| | SE | 57 | 145 | 244 | 377 |
| 27 | Average | 2058 | 4146 | 7043 | 10464 |
| | SE | 52 | 94 | 176 | 182 |

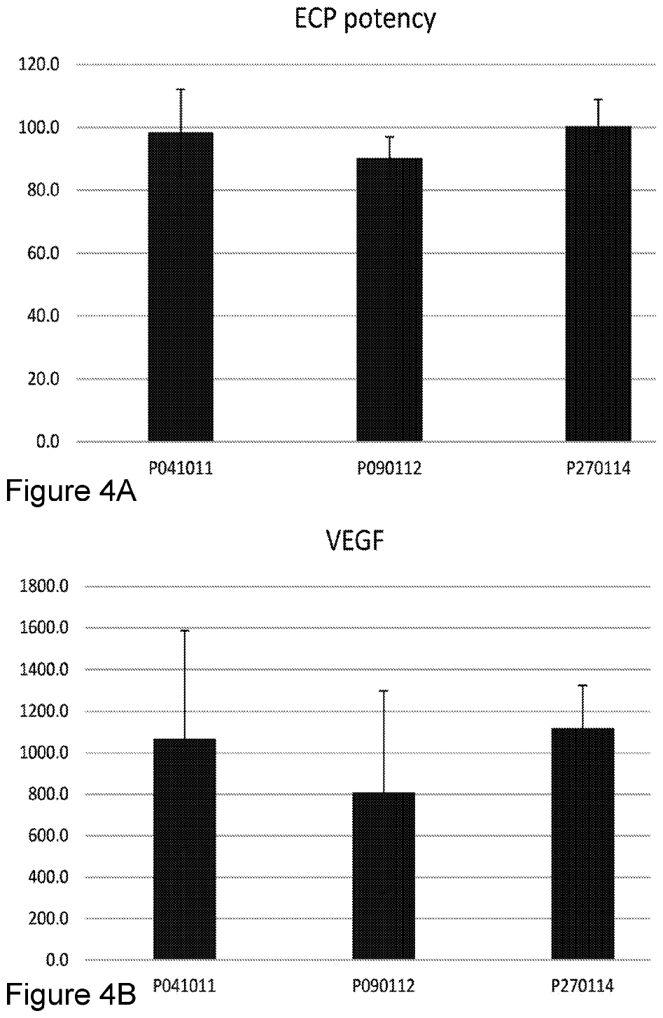

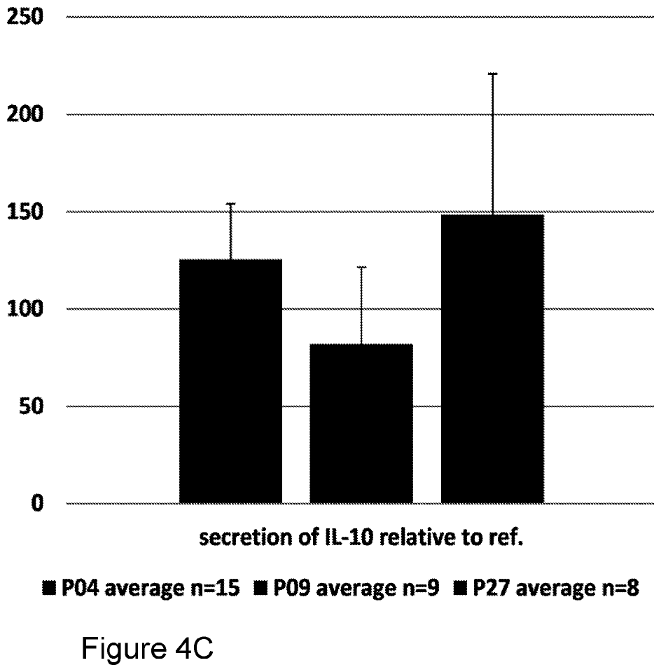

ASC from various placentas (each stored as an ICS with an unique identifier) also exhibited similar activity after 2D+3D in the ECP assay and in their VEGF secretion, as shown for 3 representative batches, P041011 (“04”), P090112 (“09”), and P270114 (“27”) (FIGS. 4A-B). In other experiments, ASC from the different placentas were incubated with U937 cells (monocytes), and IL-10 secretion from the monocytes was measured by ELISA. The different ASC populations elicited similar amounts of IL-10 secretion (FIG. 4C).

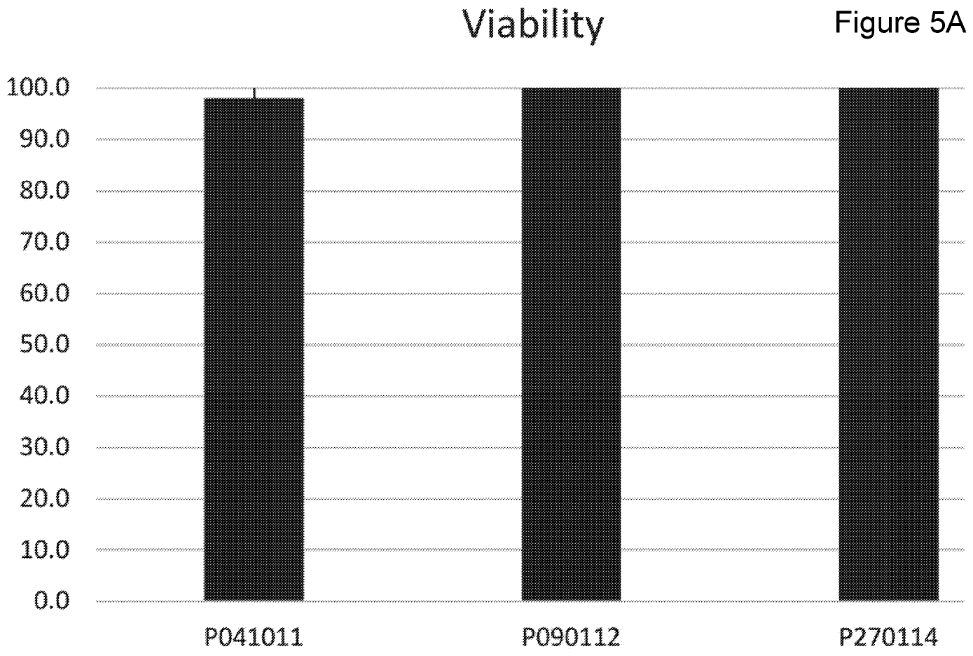

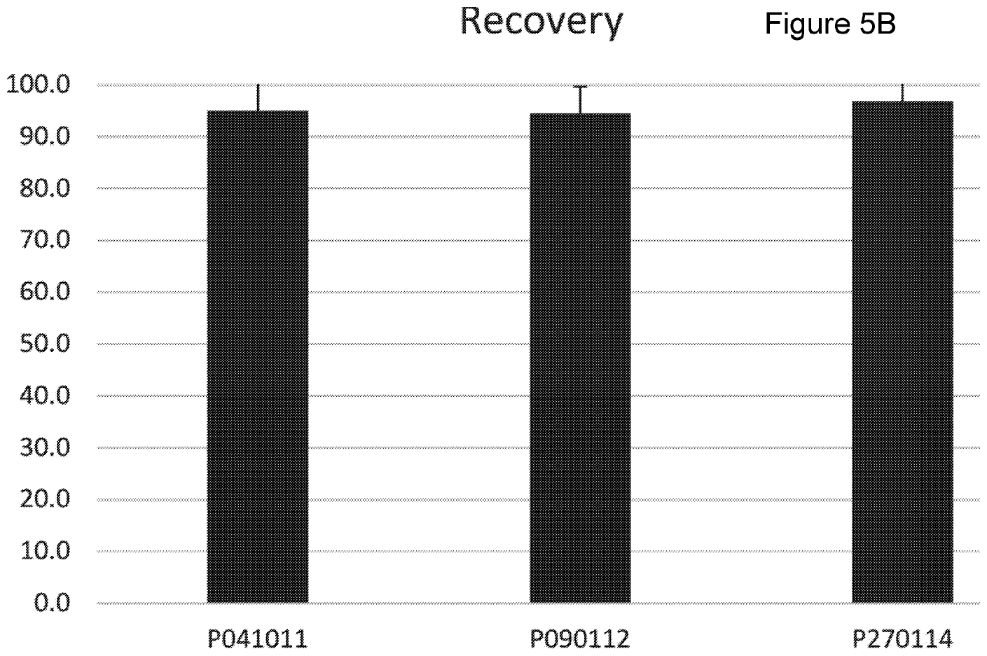

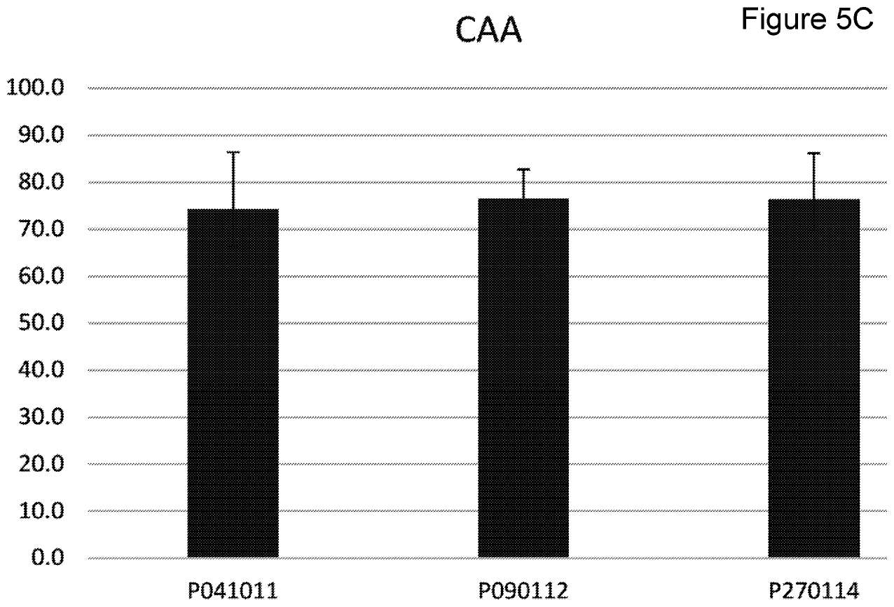

The 04, 09, and 27 batches exhibited a high degree of consistency in parameters in their percent viability, percent recovery and cell adhesion assay (FIGS. 5A-C). Levels of secreted VEGF were 800-1100 pg/ml×2 ml=1600-2200 pg. total, which can also be expressed as 1600-2200 pg. per million cells seeded.

Secretion of various cytokines by ASC from the aforementioned placentas was measured by Luminex® assays and ELISA. Specifically, Luminex® was used to measure levels of IL-6, HGF, Gro-alpha (GROa), IL-8, SDF-1 alpha, IGFBP-1, Osteoprotegerin, and Angiogenin; Angiopoietin-1, IGFBP-3, MIF, FLRG, Osteopontin, and Galectin-1; and Serpin E1, MMP-1, TIMP-1, Beta2 microglobulin, and MMP-2. ELISA was used to measure HGF, Angiogenin, and Angiopoietin-1; Decorin and Osteopontin, and; and Galectin-1 and MMP-2. The different ASC populations secreted similar amounts of the tested cytokines; the highest numbers were often not more than 2-fold the lowest numbers. Additionally, 2 batches each from the 04 and 09 placentas were tested for secretion of M-CSF, PDGF-BB, and FGF-7. For each cytokine, the highest numbers were typically not more than 2-fold the lowest numbers. ASC from different placentas also exhibited similar activity in PMBC proliferation assays, each inhibiting PMBC proliferation to a comparable extent. Results are described in PCT/IB2019/054828, to Zami Aberman et al., which is incorporated herein by reference.

In conclusion, the described methods enable production of cell populations that have different HLA types, while exhibiting a high degree of consistency in various indicators of quality and therapeutic efficacy. Similar results were obtained when ASC were prepared from different donor placentas and cultured as described in Examples 2-3.

The 04, 09, and 27 batches were used to generate the clinical data in the next Example.

### Example 5: Placental ASC Ameliorate Excess Glycation of Hemoglobin and C-Reactive Protein Levels

### Methods

### Overview:

A Phase II, multicenter, multinational, randomized, double-blind, placebo-controlled, parallel-groups study was performed, to test the safety and efficacy of placental ASC in patients with intermittent claudication (IC) due to peripheral arterial disease (PAD). The study contained 4 treatment groups:

Group #1: (“low dose”): First treatment 150×10⁶ placental ASC. Second treatment 150×10⁶ placental ASC. Group #2: (“high dose”): First treatment 300×10⁶ placental (“300M”) ASC. Second treatment 300M placental ASC. Group #3: (“placebo”): First treatment Placebo (15 mL Vehicle). Second treatment Placebo (15 mL Vehicle). Group #4: (“single treatment high dose+single treatment placebo”): First treatment 300M placental ASC. • Second treatment Placebo (15 mL Vehicle).

Approximately 170 subjects aged 45 to 85 years and diagnosed with IC due to PAD were enrolled, as follows: 37, 48, 50, and 37 patients treated in Groups 1-4, respectively. 33, 42, 45, and 33 patients, respectively, were included in the mFAS (described below).

Subjects received the assigned treatment twice to the affected leg, with a 12-week interval between each treatment.

The study contained 6 stages:
1. Screening Period of up to 4 weeks.
2. First treatment of placental ASC or placebo at week 0.
3. Short-term follow-up at 24 hours after first treatment, and weeks 1 and 4 after first treatment.
4. Repeat dose of placental ASC or placebo at week 12 after first treatment.
5. Short-term follow-up 24 hours after second treatment.
6. Long-term follow-up at weeks 13, 26, 39 and 52 after first treatment. Study termination at week 65 after first treatment.

Detailed Methodology:

Stage I, Screening Period: Week −4-0

The screening period included screening number assignment and diagnosis confirmation.

Inclusion/Exclusion assessment, including two baseline Exercise Treadmill Tests (ETTs) performed with a time interval of 7-10 days, demographic information, and medical history, including an allergy history questionnaire and concomitant medication. A washout period of at least 2 weeks was observed from vasodilators prescribed for IC prior to the first ETT. Vital signs, physical examination, ECG, ABI and/or TBI, and laboratory tests (serum pregnancy test, hematology, blood chemistry, urinalysis and coagulation profile) were collected and analyzed.

After eligibility was confirmed, subjects were randomized to receive 1 of 2 target doses of placental ASC or placebo at least 1 week prior to the planned first treatment.

Stage II, Visit La (First Dose Treatment): Week 0

On the first treatment day and prior to the placental ASC/placebo treatment, the following were performed: Inclusion/Exclusion re-assessment, vital signs, ECG, resting ABI and/or TBI, laboratory tests (urine pregnancy test [for women of child-bearing potential], urinalysis, hematology, blood chemistry, IL-6 testing for a subset of the subjects (immunology profile), HLA-typing, HLA-Abs and tryptase levels were collected. Two health-related Quality of Life Questionnaires (QoL SF-36v2 and Peripheral Arterial Questionnaire (PAQ)) were administered. AEs and concomitant medications were recorded.

Antihistamine pre-treatment was given 1 hour (±15 minutes) prior to the study treatment administration to ensure coverage for 24 hours and as long as necessary post study treatment administration.

Pre-medication with analgesics was administered to the subject at the Investigator's discretion as long, as it did not require cardio-respiratory monitoring of the subject.

Upon satisfactory completion of the pre-treatment steps, placental ASC/placebo was administered after 30 minutes of rest via 30 intramuscular injections delivered to the most affected leg. The most affected leg was defined as the leg with the lowest ABI and/or TBI at screening. However, in cases where the leg with the lowest ABI and/or TBI at screening was not the most symptomatic leg (i.e. the leg that limits the subject's walking), then the investigator injected the most symptomatic leg according to his clinical judgment, as long as it fulfilled the ABI and/or TBI inclusion criteria.

After a 1-hour monitoring period, vital signs were measured and AEs were recorded.

If an allergic/hypersensitivity reaction occurred while the subject was still under medical assessments, blood samples for tryptase values were collected immediately. For subjects developing an allergic/hypersensitivity reaction following discharge, additional tryptase blood samples were collected within 4 hours of the first appearance of the allergic/hypersensitivity reaction, or as soon as possible thereafter.

Stage III, Visits Lb 2 and 3 (Short Term Follow Up): 24 Hours after First Dose Treatment

Weeks 1 and 4

Visit 1b: 24 hours after initial treatment, AEs, vital signs (including pulse oximetry measurement), and physical examination were performed, and blood samples for tryptase levels were collected.

Visit 2: week 1 after initial treatment, vital signs, ECG, AEs, concomitant medication and laboratory blood tests [(hematology, blood chemistry, IL-6 measurement, and HLA-Abs] were collected and analyzed.

Visit 3: week 4 after initial treatment, vital signs, AEs, concomitant medication and laboratory blood tests (hematology, blood chemistry) were collected and analyzed.

Stage IV, Visit 4a (Repeat Dose Treatment): Week 12

On the day before the second dosing, vital signs, ECG, resting ABI and/or TBI, health-related QoL Questionnaires, ETT, AEs, concomitant medication, and laboratory tests (urine pregnancy test, urinalysis, hematology, blood chemistry, and IL-6 measurement) and HLA-Abs and tryptase levels were collected and analyzed.

If any subjects developed a severe allergic/hypersensitivity reaction that required hospitalization and/or treatment with intravenous steroids/epinephrine following visit 1a, or for whom, in the opinion of the investigator, the risk of developing such severe allergic/hypersensitivity reactions increased since the screening, they were contraindicated from receiving the second dosing.

The second dosage was given in the same affected leg treated at visit 1a. The dosage and immediate follow-up protocols were essentially identical to the first dosing.

Stage V, Visit 4b (Short Term Follow Up): 24 Hours after Repeated Dose Treatment

Visit 4b, 24 hours after second treatment: AEs, vital signs (including pulse oximetry measurement), physical examination and blood samples for tryptase levels were collected and analyzed.

Stage VI (Long Term Follow Up): Weeks 13, 26, 39 and 52, Termination Visit (Week 65) and Unscheduled Visit

During the long term follow up period, subjects visited the Medical Center on weeks 13, 26, 39, 52 and 65 (termination visit) for follow-up by a clinical Investigator.

Visit 5, week 13: One week (±1 day) after the second treatment, vital signs, ECG, AEs, concomitant medication and laboratory tests (hematology, chemistry, IL-6 measurement) and HLA-Abs were collected and analyzed.

Visit 6, week 26: vital signs, resting ABI and/or TBI, 2 health-related QoL Questionnaires, ETT, AEs and concomitant medication were recorded.

Visit 7, week 39: vital signs, resting ABI and/or TBI, 2 health-related QoL Questionnaires, ETT, AEs and concomitant medication and laboratory tests (hematology and chemistry) were collected and analyzed.

Visit 8, week 52: vital signs, resting ABI and/or TBI, 2 health-related QoL Questionnaires, ETT, AEs and concomitant medication and laboratory tests (hematology and chemistry) were collected and analyzed.

Termination Visit, week 65/early discontinuation: vital signs, physical examination, ECG, resting ABI and/or TBI, 2 health-related QoL Questionnaires, ETT, AEs, concomitant medication, and laboratory tests (urine pregnancy test [women of child-bearing potential], urinalysis, hematology, HLA-Abs and blood chemistry) were collected and analyzed.

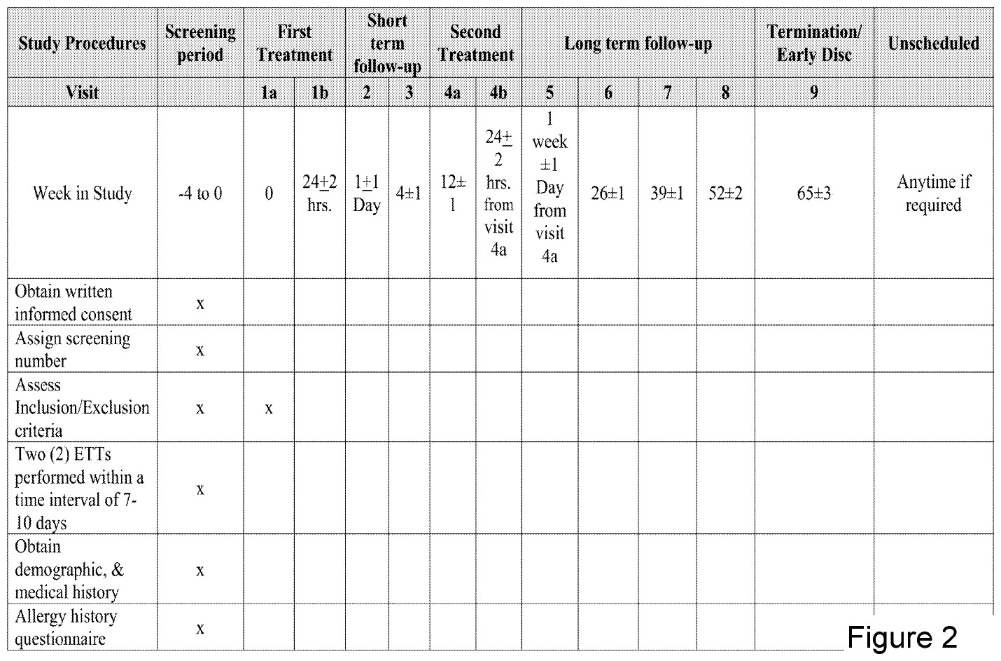

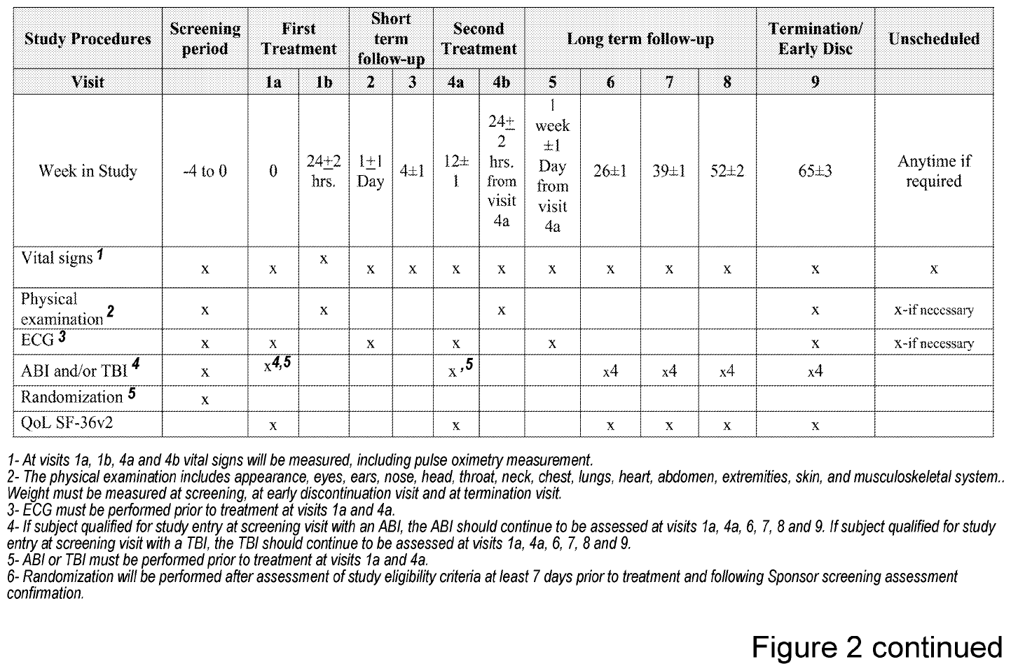

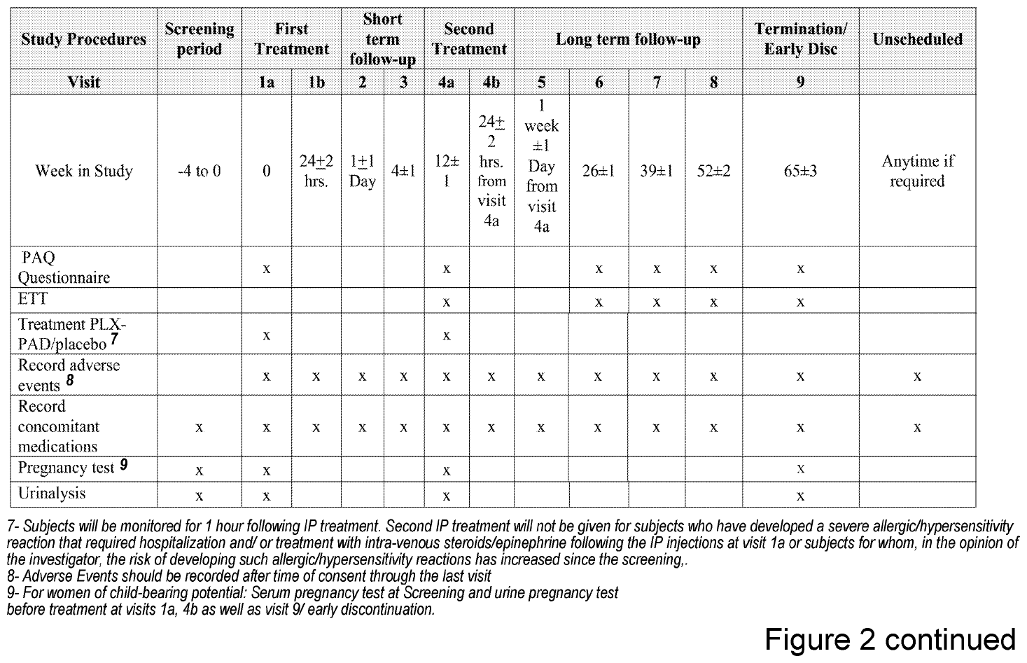

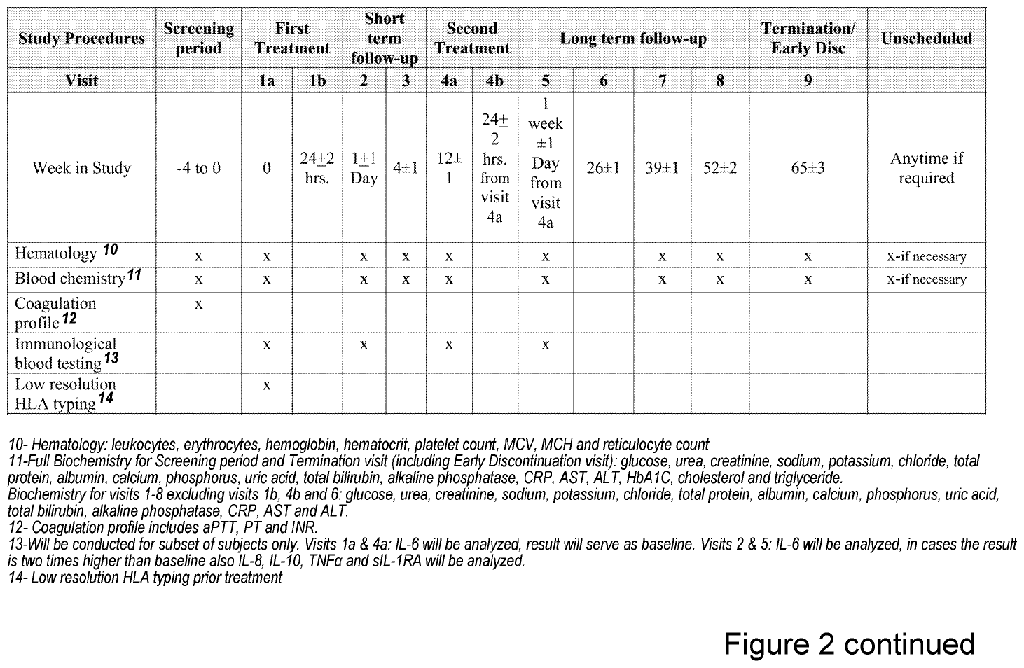

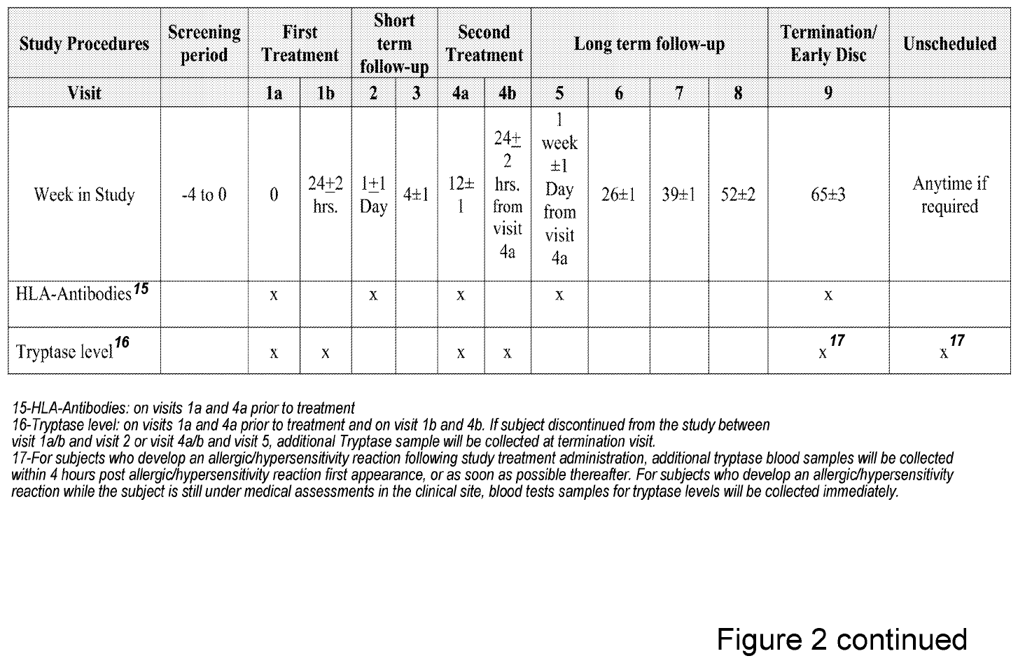

The study visit flow chart is depicted in FIG. 2.

For immunological profile, blood samples were tested for levels of IL-6, IL-8, IL-10, TNF-α, and sIL-1RA where applicable.

Safety Endpoints:
Treatment emergent adverse events, SAEs, AEs leading to premature study termination.
Safety laboratory values
Immunological reaction
Major Amputation of the Lower Extremity
Death rates

Efficacy Endpoints:

Primary Endpoint:
Log ratio of week 52 MWD to baseline MWD

Secondary Endpoints:
Log ratio of week 52 ICD to baseline ICD.
Change from baseline to Week 52 in Peripheral Arterial Questionnaire (PAQ)
Change from baseline to Week 52 in Quality of Life (QoL) Questionnaire (SF-36v2)
Change—baseline to Week 52 in hemodynamic measurements (resting ABI and/or TBI)
Revascularization rates at week 52.

Study Population

This study was conducted in subjects aged 45-85 years and diagnosed with IC due to PAD.

Inclusion and exclusion criteria are described in PCT/IB2019/054828, to Zami Aberman et al, which is incorporated herein by reference.

Assessment of Ankle-Brachial Index (ABI)/Toe-Brachial Index (TBI), exercise treadmill tests, and assays for post-intervention cellular immune response are described in PCT/IB2019/054828, to Zami Aberman et al, which is incorporated herein by reference.

Drug Randomization

All subjects entered into the study were randomized to receive either placental ASC or placebo, using a blocked randomization procedure. In order to safeguard the double-blind nature of the study, the staff members handling the treatment were not allowed to perform the screening and follow-up visits.

Blinding

Except the unblinded staff members handling the treatment, all investigators and any personnel involved in the subject's assessment, monitoring, analysis, and data management (excluding the designated personnel), were blinded to the subject assignment. In the event of an SAE or pregnancy, when study drug assignment was needed to make treatment decisions for the subject, the investigator was allowed to unblind the subject's drug assignment. In any case, the subject's drug code assignment was not revealed to the sponsor.

The efficacy analysis was performed at 52 weeks, and the primary and secondary efficacy analyses utilized the data collected until week 52.

Analysis Sets for the First Stage of Efficacy Analysis (52 Weeks)

Intent-to-Treat Analysis Set (ITT)

The intent-to-treat (ITT) analysis set includes all randomized patients. In this population, treatment was assigned based on the treatment to which patients were randomized, regardless of which treatment they actually received. The ITT analysis set includes efficacy observations that were measured up to week 52. Due to a temporary regulatory concern during the trial, which prevented some patients from receiving the second treatment, this analysis set was used for exploratory purposes only.

Full Analysis Set (FAS)

The Full Analysis Set (FAS) includes subjects in the ITT analysis set, who received at least one study treatment, and have at least 1 post baseline usable treadmill assessment. The FAS analysis set includes efficacy observations that were measured up to week 65.

Modified Full Analysis Set (mFAS)

The modified full analysis set (mFAS) includes all patients in the ITT analysis set who received at least 1 treatment and had at least 1 post baseline treadmill assessment, excluding those that did not receive the 2nd treatment due to the aforementioned concern. The mFAS analysis set includes efficacy observations that were measured up to week 52.

Full Analysis Set-Subjects that Received 2 Study Treatments (FAS2Rx)

The full analysis set for subjects that received 2 study treatments (FAS2Rx) includes subjects in the FAS analysis set, excluding all subjects that did not receive the 2nd study treatment from any reason (a total of 26 subjects). The FAS2Rx analysis set includes efficacy observations that were measured up to week 65.

Primary Efficacy End-Point and Analysis

The primary endpoint for this study is log ratio of week 52 MWD to baseline MWD. The principal analysis of the primary endpoint utilizes the Mixed Model for Repeated Measures (MMRM) (SAS® MIXED procedure with REPEATED sub-command). The model includes the following fixed effects: categorical week in trial by treatment interaction, site, and log of baseline MWD measurement. The model uses the unstructured covariance structure and the REML estimation method, and degrees of freedom are adjusted using the Kenward-Roger method. Data from all post-baseline to baseline log ratios visits was used as response in the model, and differences between the treatments groups at week 52 were estimated using contrasts.

Results

Different doses, schedules, and batches of placental ASC were tested in patients with intermittent claudication, as below: Group #1: (“low dose”): First treatment 150×10⁶ placental ASC. Second treatment 150×10⁶ placental ASC. Group #2: (“high dose”): First treatment 300×10⁶ placental ASC. Second treatment 300×10⁶ placental ASC. Group #3: (“placebo”): First treatment Placebo (15 mL Vehicle). Second treatment Placebo (15 mL Vehicle). Group #4: (“single treatment high dose+single treatment placebo”) First treatment 300×10⁶ placental ASC. Second treatment Placebo (15 mL Vehicle).

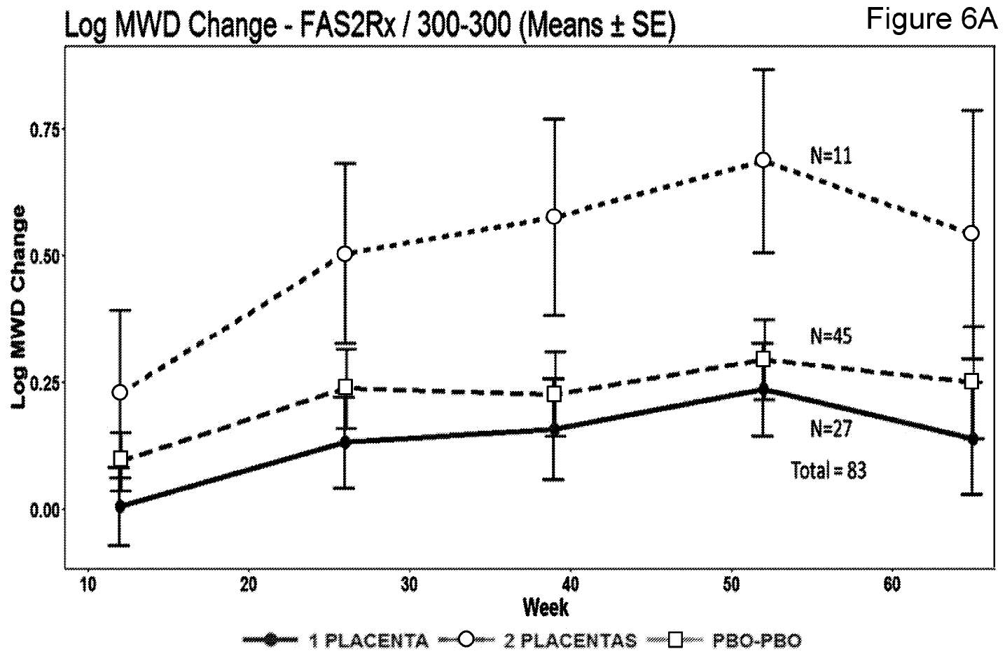

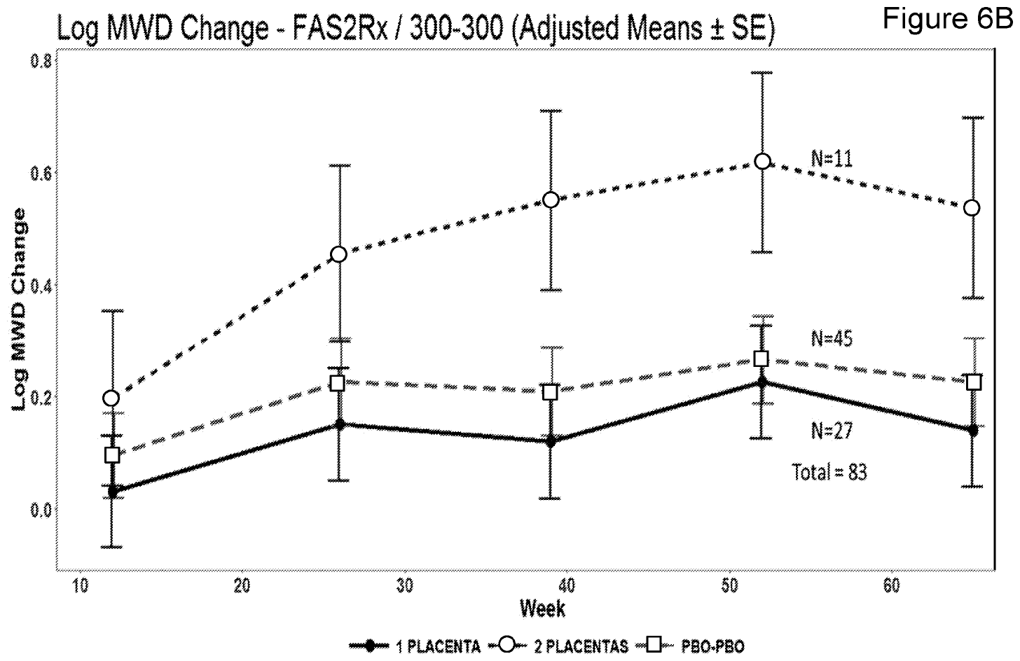

Overall, a positive therapeutic effect on IC was observed relative to placebo, particularly with the 300M-cell dose. Considering the subjects who received the 300M dose, 2 injections was superior to a single injection (FIG. 6).

Moreover, a significant reduction in Hemoglobin A1C (HbA1C) was observed in subjects that received either 1 or 2 doses of 300M ASC. The reduction was even sharper in subjects who received ASC from two different placentas (Table 4). Subjects in the different groups had similar baseline HbA1C values (Table 5)

TABLE 4
Week 65 ANCOVA of Change from Baseline in HbAlC
(mmol/mol). Difference of Adjusted Means

| | | | | Lower | Upper |
|---|---|---|---|---|---|
| | | | P- | 95% CI | 95% CI |
| Comparison | Estimate | SE | Value | Limit | Limit |
| 300M-PBO- | −4.414 | 1.978 | 0.0273 | −8.326 | −0.503 |
| PBO-PBO | | | | | |
| 150M-150M- | 0.740 | 0.200 | 0.1399 | 0.495 | 1.107 |
| PBO-PBO | | | | | |
| 300M-300M- | −2.147 | 1.941 | 0.2706 | −5.986 | 1.691 |
| PBO-PBO | | | | | |
| 300M-300M | −7.770 | 3.087 | 0.0155 | −13.988 | −1.553 |
| different | | | | | |
| placentas- | | | | | |
| PBO-PBO | | | | | |

TABLE 5
Baseline HbAlC values in the study groups.

| Group | N | Mean | SD |
|---|---|---|---|
| PBO-PBO | 43 | 46.13 | 11.75 |
| 300M-PBO | 30 | 43.93 | 9.90 |
| 150M-150M | 32 | 43.38 | 9.84 |
| 300M-300M | 40 | 44.34 | 8.71 |

300M-300M subgroups (only subjects with HbAlc data at wk 65)

| | | | |
|---|---|---|---|
| 300M-300M different placentas | 11 | 48.1 | 7.7 |
| 300M-300M same placentas | 23 | 43.4 | 11.1 |

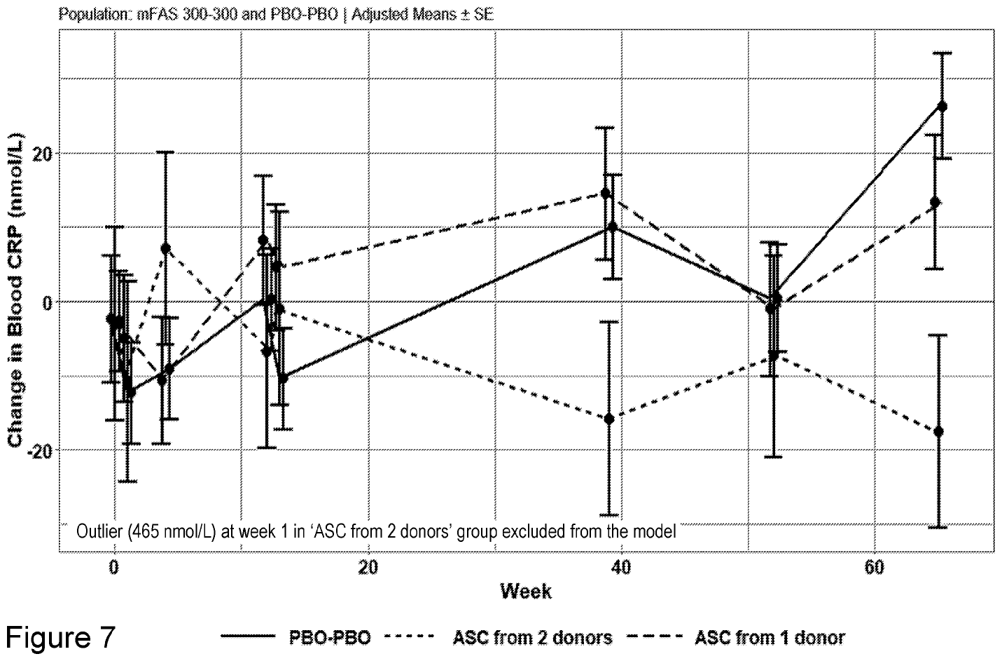

Additionally, subjects who received ASC from two different placentas exhibited a reduction from baseline CRP levels, which was not seen in the PBO group (FIG. 7 and Table 6).

TABLE 6
Week 65 Descriptive Statistics of Change
from Baseline in Blood CRP (nmol/L).

| | | Treatment | | |
|---|---|---|---|---|
| | | 300M-300M | 300M-300M | |
| | | from same | from | PBO- |
| | | donor | different donors | PBO |
| Baseline | N | 24 | 11 | 40 |
| | Mean | 28.4 | 45.5 | 32.9 |
| | SD | 27.5 | 47.4 | 38.8 |
| | Min | 0.1 | 0.1 | 0.1 |
| | Median | 17.7 | 28.6 | 22.4 |
| | Max | 111.4 | 151 | 189.3 |
| Change from | N | 24 | 11 | 40 |
| Baseline | Mean | 13.8 | -7.5 | 29 |
| | SD | 67.9 | 56.8 | 95 |
| | Min | −41.9 | −125.9 | −58.1 |
| | Median | −0.2 | −1.4 | 1.4 |
| | Max | 297.6 | 83 | 521.1 |

Additionally, the safety profile of the placental ASC was excellent. Most categories of adverse events were either unaffected or reduced (Table 7).

TABLE 7
Adverse events in the study groups.

| | PBO- | 300- | 150- | 300- |
|---|---|---|---|---|
| | PBO | PBO | 150 | 300 |
| | (n = 51) | (n = 36) | (n = 37) | (n = 48) |
| Death | 0% | 0% | 0% | 2.1% |
| Major amputations | 3.9% | 0% | 0% | 0% |
| Malignancies | 7.8% | 5.6% | 10.8% | 2.1% |
| Infections | 33.3% | 22.2% | 32.4% | 33.3% |
| Injection site pain | 39.2% | 30.6% | 40.5% | 47.9% |
| hematoma | 9.8% | 2.8% | 5.4% | 6.3% |
| → leading to discontinuation | 0% | 2.8% | 2.7% | 4.2% |
| Peripheral vascular disorders | 29.4% | 27.8% | 27.0% | 22.9% |
| Cardiac disorders | 9.8% | 11.1% | 8.1% | 6.3% |
| Neurologic disorders | 27.5% | 13.9% | 18.9% | 20.8% |
| Blood and lymphatic disorders | 9.8% | 8.3% | 2.7% | 2.1% |
| Renal disorders | 9.8% | 8.3% | 5.4% | 6.3% |
| Ophthalmologic disorders | 11.8% | 5.6% | 5.4% | 4.2% |
| Respiratory tract disorders | 17.6% | 2.8% | 10.8% | 8.3% |
| Abnormal lab findings | 13.7% | 8.3% | 8.1% | 6.3% |
| Gastrointerstinal disorders | 23.5% | 25.0% | 18.9% | 20.8% |
| Musculosceletal disorders | 39.2% | 41.7% | 35.1% | 31.1% |
| Psychiatric disorders | 7.8% | 5.6% | 0% | 4.2% |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

### Additional References May be Cited in Text

Alelign T et al., Kidney Transplantation: The Challenge of Human Leukocyte Antigen and Its Therapeutic Strategies. J Immunol Res. 2018 Mar. 5; 2018:5986740. doi: 10.1155/2018/5986740. eCollection 2018.
Biyani R S et al., Algorithms for red lesion detection in Diabetic Retinopathy: A review. Biomed Pharmacother. 2018 November; 107:681-688. doi: 10.1016/j.biopha.2018.07.175. Epub 2018 Aug. 18.
Chin et al., “Clinical usefulness of a novel Clq assay to detect immunoglobulin G antibodies capable of fixing complement in sensitized pediatric heart transplant patients,” The Journal of Heart and Lung Transplantation, vol. 30, no. 2, pp. 158-163, 2011.
Clayton A et al., Analysis of antigen presenting cell derived exosomes, based on immuno-magnetic isolation and flow cytometry. J Immunol Methods. 2001; 247(1-2):163-74.
Conde-Vancells J., Rodriguez-Suarez E., Gonzalez E., Berisa A., Gil D., Embade N., Valle M., Luka Z., Elortza F., Wagner C., Lu S. C., Mato J. M., Falcon-Perez M. (2010) Candidate biomarkers in exosome-like vesicles purified from rat and mouse urine samples. Proteomics Clin. Appl. 4, 416-425.
Crescitelli R et al., Distinct RNA profiles in subpopulations of extracellular vesicles: apoptotic bodies, microvesicles and exosomes. J Extracell Vesicles. 2013 Sep. 12; 2.
George C et al., Increased risk of respiratory diseases in adults with Type 1 and Type 2 diabetes. Diabetes Res Clin Pract. 2018 August; 142:46-55. doi: 10.1016/j.diabres.2018.05.029. Epub 2018 May 24.
Gupta S1 et al., Biomarkers of Systemic Inflammation and Risk of Incident Hearing Loss. Ear Hear. 2019 July/August; 40(4):981-989. doi: 10.1097/AUD.0000000000000678.
Khaw K T et al., Association of hemoglobin Alc with cardiovascular disease and mortality in adults: the European prospective investigation into cancer in Norfolk. Ann Intern Med. 2004 Sep. 21; 141(6):413-20. PMID: 15381514.
Koga K., Matsumoto K., Akiyoshi T., Kubo M., Yamanaka N., Tasaki A., Nakashima H., Nakamura M., Kuroki S., Tanaka M., Katano M. (2005) Purification, characterization and biological significance of tumor-derived exosomes. Anticancer Res. 25, 3703-3707.
Kannagi R et al., Stage-specific embryonic antigens (SSEA-3 and −4) are epitopes of a unique globo-series ganglioside isolated from human teratocarcinoma cells. EMBO J. 1983; 2(12):2355-61.
Kinzebach S and Bieback K. Expansion of Mesenchymal Stem/Stromal cells under xenogenic-free culture conditions. Adv Biochem Eng Biotechnol. 2013; 129:33-57.
Mathias R A et al., Isolation of extracellular membranous vesicles for proteomic analysis. Methods Mol Biol. 2009; 528:227-42.
Nathan D M et al (Diabetes Control and Complications Trial Research Group), The effect of intensive treatment of diabetes on the development and progression of long-term complications in insulin-dependent diabetes mellitus. N Engl J Med. 1993 Sep. 30; 329(14):977-86.
Nilsson J et al., (2009) Prostate cancer-derived urine exosomes: A novel approach to biomarkers for prostate cancer. Br. J. Cancer 100, 1603-1607
Palmisano G et al., Characterization of membrane-shed microvesicles from cytokine-stimulated-cells using proteomics strategies. Mol Cell Proteomics. 2012 August; 11(8):230-43. doi: 10.1074/mcp.M111.012732.
Robinson J, Halliwell J A, Hayhurst J H, Flicek P, Parham P, Marsh S G E: The IPD and IMGT/HLA database: allele variant databases. Nucleic Acids Research. 2015 43:D423-431.
Selvin E et al., Meta-analysis: glycosylated hemoglobin and cardiovascular disease in diabetes mellitus. Ann Intern Med. 2004 Sep. 21; 141(6):421-31.
Sidney J, Peters B, Frahm N, Brander C, Sette A. HLA class I supertypes: a revised and updated classification. BMC Immunol. 2008 Jan. 22; 9:1. doi: 10.1186/1471-2172-9-1.
Silva et al., “Evaluation of HLA matchmaker compatibility as predictor of graft survival and presence of anti-HLA antibodies,” Transplantation Proceedings, vol. 42, no. 1, pp. 266-269, 2010.
Umanath K et al., Update on Diabetic Nephropathy: Core Curriculum 2018. Am J Kidney Dis. 2018 June; 71(6):884-895. doi: 10.1053/j.ajkd.2017.10.026.
Valenzuela and Reed, “Antibodies in transplantation: the effects of HLA and non-HLA antibody binding and mechanisms of injury,” in Transplantation Immunology. Methods in Molecular Biology (Methods and Protocols), vol. 1034, pp. 41-70, Humana Press, Totowa, N.J., USA, 2013.

What is claimed is:

1. A method of ameliorating impaired glucose tolerance in a subject in need thereof, comprising: administering to the subject a pharmaceutical composition, comprising placental adherent stromal cells (ASC), thereby ameliorating impaired glucose tolerance.

2. The method of claim 1, wherein said subject is at least 60 years of age at the onset of treatment.

3. The method of claim 1, wherein said subject has a HbA1c value of at least 43.5 mmol/mol at the onset of treatment.

4. The method of claim 1, wherein said subject has a body mass index of at least 27.5 kg/m² at the onset of treatment.

5. A method of reducing systemic inflammation in a subject with impaired glucose tolerance in a subject in need thereof, comprising: administering to the subject a pharmaceutical composition, comprising placental adherent stromal cells (ASC), thereby reducing systemic inflammation in a subject with impaired glucose tolerance.

6-8. (canceled)

9. The method of claim 1, wherein said ASC have been incubated on a 3D culture substrate in a bioreactor.

10. The method of claim 9, further comprising the subsequent step of harvesting said ASC by removing said ASC from said 3D culture apparatus.

11. The method of claim 9, wherein said ASC have been incubated on a 2D adherent-cell culture substrate, prior to incubation in said 3D culture apparatus.

12. (canceled)

13. The method of claim 9, wherein said 3D culture substrate comprises a synthetic adherent material formed as a fibrous matrix, wherein said synthetic adherent material is selected from the group consisting of a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber.

14-16. (canceled)

17. The method of claim 1, wherein said administering comprises:
a. administering to the subject a first pharmaceutical composition, comprising allogeneic placental ASC from a first donor; and
b. administering to said subject, at least 7 days after step a), a second pharmaceutical composition comprising allogeneic placental ASC from a second donor, wherein said second donor differs from said first donor in at least one allele group of human leukocyte antigen (HLA)-A or human leukocyte antigen (HLA)-B.

18. The method of claim 17, further comprising administering to said subject, at least 7 days after step b), a third pharmaceutical composition comprising allogeneic ASC of a third donor, wherein said third donor differs from both said first donor and said second donor in at least one allele group of HLA-A or HL A-B.

19. The method of claim 1, wherein said ASC express a marker selected from the group consisting of CD73, CD90, CD29 and CD105.

20. The method of claim 19, wherein said ASC do not express a marker selected from the group consisting of CD3, CD4, CD11b, CD14, CD19, and CD34.

21. The method of claim 19, wherein said ASC do not express a marker selected from the group consisting of CD3, CD4, CD34, CD39, and CD106.

22. (canceled)

23. The method of claim 21, wherein more than 50% of said ASC express CD200.

24. The method of claim 21, wherein more than 50% of said ASC express CD141.

25. The method of claim 21, wherein more than 50% of said ASC express SSEA4.

26. The method of claim 25, wherein said ASC secrete Flt-3 ligand or stem cell factor (SCF).

27. The method of claim 1, wherein the cells are administered intramuscularly.

28. The method of claim 1, wherein the cells are administered intravenously, subcutaneously, or intraperitoneally.
